(19) United States
(12) Patent Application Publication
(10) Pub. No.: US 2015/0150787 A1
(43) Pub. Date: Jun. 4, 2015

(54) COMPOSITIONS, METHODS & SYSTEMS FOR RESPIRATORY DELIVERY OF THREE OR MORE ACTIVE AGENTS

(71) Applicant: Pearl Therapeutics, Inc., Redwood City, CA (US)

(72) Inventors: David Lechuga-Ballesteros, San Jose, CA (US); Vidya B. Joshi, Redwood City, CA (US); Colin Reisner, Randolph, NJ (US); Patrick FitzGerald Darken, Maple Glen, PA (US); Sarvajna Dwivedi, Redwood City, CA (US)

(73) Assignee: Pearl Therapeutics, Inc., Redwood City, CA (US)

(21) Appl. No.: 14/285,435

(22) Filed: May 22, 2014

Related U.S. Application Data
(60) Provisional application No. 61/826,424, filed on May 22, 2013.

(51) Int. Cl. A61K 9/00; A61K 31/40; A61K 47/02; A61K 47/06; A61K 47/24; A61K 31/137; A61K 31/58

(52) U.S. Cl. CPC A61K 9/008; A61K 31/137; A61K 31/40; A61K 31/58; A61K 47/06; A61K 47/24; A61K 47/02

(57) ABSTRACT

Pharmaceutical compositions, systems and methods suitable for respiratory delivery of a fixed combination of LAMA, LABA, and ICS active agents are described. The pharmaceutical compositions described herein may be formulated for respiratory delivery via a metered dose inhaler (MDI). Also described herein are MDI systems for delivery of a fixed combination of LAMA, LABA, and ICS active agents, as well as methods for preparing and using the compositions and systems described herein.

### TECHNICAL FIELD

The present disclosure relates generally to compositions, methods and systems suitable for respiratory delivery of three or more active agents. In certain embodiments, the present disclosure relates to compositions, methods, and systems suitable for respiratory delivery of three active agents, wherein the active agents include a long-acting muscarinic antagonist (LAMA), a long-acting β₂ adrenergic agonist (LABA), and an inhaled corticosteroid (ICS).

### BRIEF DESCRIPTION OF THE DRAWINGS

FIG. 1 provides cascade impaction data for mometasone furoate delivered from three different triple cosuspension compositions described in Example 1.

FIG. 2 provides a graph illustrating the fine particle mass (FPM) of mometasone furoate, glycopyrrolate, and formoterol fumarate delivered from three different triple cosuspension compositions described in Example 1.

FIGS. 3A-C provide cascade impaction data for mometasone furoate, glycopyrrolate, and formoterol fumarate delivered from cosuspenion compositions described in Example 1, with FIG. 3A providing cascade impaction data for the composition formulated to provide a delivered dose of 100 μg mometasone furoate per MDI actuation, FIG. 3B providing cascade impaction data for the composition formulated to provide a delivered dose of 200 μg mometasone furoate per MDI actuation, and FIG. 3C providing cascade impaction data for the composition formulated to provide a delivered dose of 300 μg mometasone furoate per MDI actuation.

FIGS. 4A-C provide cascade impaction data illustrating dose linearity for each of the mometasone furoate, glycopyrrolate, and formoterol fumarate actives delivered from three different triple cosuspension compositions described in Example 1, with FIG. 4A providing cascade impaction data for mometasone furoate delivered from each of the three compositions, FIG. 4B providing cascade impaction data for formoterol fumarate delivered from each of the three compositions, and FIG. 4C providing cascade impaction data for glycopyrrolate delivered from each of the three compositions.

FIG. 5 provides cascade impaction profiles and aerosol characteristics for formoterol fumarate delivered from the GFF, BGF1, and BGF2 compositions described in Example 2.

FIG. 6 provides cascade impaction profiles and aerosol characteristics for budesonide delivered from the BGF1, BGF2, and BGF3 compositions described in Example 2.

FIG. 7 provides cascade impaction profiles and aerosol characteristics for glycopyrronium delivered from the GFF, BGF1, and BGF2 compositions described in Example 2.

FIG. 8 provides cascade impaction profiles and aerosol characteristics for budesonide delivered from the BGF3, BD Mono, and BFF compositions described in Example 2.

FIG. 9 provides a graph illustrating the geometric mean plasma concentration over time of budesonide administered to patients as part of a clinical trial using various formulations, including triple cosuspension compositions according to the present description.

FIG. 10 provides a graph illustrating the geometric mean plasma concentration over time of glycopyrronium administered to patients as part of a clinical trial using various formulations, including triple cosuspension compositions according to the present description.

FIG. 11 provides a graph illustrating the geometric mean plasma concentration over time of formoterol administered to patients as part of a clinical trial using various formulations, including triple cosuspension compositions according to the present description.

### DETAILED DESCRIPTION

The present disclosure provides pharmaceutical compositions, systems and methods suitable for respiratory delivery of three or more active agents via an MDI. In certain embodiments, at least one of the active agents is selected from LAMA, LABA, and ICS agents. In more particular embodiments, the pharmaceutical compositions described herein include three active agents including a LAMA active agent, a LABA active agent, and an ICS active agent. The pharmaceutical compositions described herein may be formulated for respiratory delivery via an MDI. Also described herein are MDI systems for delivery of three or more active agents, as well as methods for preparing the compositions and systems described herein.

Pulmonary diseases, such as chronic obstructive pulmonary disease (“COPD”) and asthma, are one of the leading causes of death in most countries, and the prevalence of pulmonary disease is increasing. Pulmonary diseases are typically characterized by a limitation of airflow into and/or within the lungs, and they are often multicomponent diseases. In the case of COPD, the diminished lung capacity is generally progressive and, using available treatments, not fully reversible. Patients suffering from pulmonary disease may also experience acute exacerbations of their condition, particularly in the later stages of a progressive disease. Such acute exacerbations can have significant, negative impacts on the patient's quality of life and ability to participate in daily activities. The effects of pulmonary diseases and disorders can even vary throughout each day. For example, patients with COPD report that their symptoms, including severe shortness of breath and the accompanying limitation on physical activity, are most problematic in the mornings.

Therapeutic approaches that utilize a combination of active agents may provide clinical benefits additional to those associated with each active agent alone. In particular, combination therapies that utilize LAMA, LABA, and ICS active agents may provide improved long-term management of moderate to severe pulmonary disease. A pharmaceutical formulation and delivery system capable of respiratory delivery of a fixed combination of LAMA, LABA, and ICS active may provide the therapeutic benefits available from a therapeutic regimen including all three classes of active agent, while also working to increase patient convenience and compliance. However, to gain combination product approval in the context of respiratory delivery, active agents in fixed combination products are expected to have comparable aerosol and deliverability properties (e.g., as measured in vitro by cascade impactor aerodynamic profiles) to monotherapy products of the same active agents, such that the potential clinical performance of the combination can be assessed relative to its component active agents, without confounding effects due to drug delivery differences, which are often introduced by combining active agents (that is, without a coformulation effect).

As used herein, the term “fixed combination” refers to a combination of three or more active agents included within a single pharmaceutical formulation such that each of the three or more active agents are delivered simultaneously upon administration of the pharmaceutical formulation. In particular embodiments, the pharmaceutical formulations described herein are suspension formulations that include a fixed combination of a LAMA active agent, a LABA active agent, and an ICS active agent and are suitable for respiratory delivery of the combined active agents to a patient via a metered dose inhaler (“MDI”).

Formulating pharmaceutical compositions incorporating three or more active agents is challenging due to unpredictable or unexpected interactions between the active agents or changes to the formulations resulting from the incorporation of additional active agents. Such interactions are generally known as “coformulation effects” or a “coformulation effect.” In the context of suspension formulations delivered from an MDI, a coformulation effect may be manifest by, for example, a deviation from similarity between a formulation including a single active agent and a formulation including a combination of two or more active agents in one or more of the following areas: aerosol and/or particle size distribution characteristics provided by the formulation; delivered dose uniformity for one or more of the active agents; deliverability or absorption of one or more of the active agents; and the dose proportionality observed for one or more of the active agents. Drug-drug interactions are a type of coformulation effect that can be particularly challenging to overcome. As used herein, “drug-drug interaction” refers to a change to the effect of a first drug when the first drug is administered with one or more additional drugs. The change resulting from a drug-drug interaction may be an increase or a decrease in the action of the drug, a change in the rate of absorption of the drug, a change to the quantity of drug absorbed in the body, or other changes to the pharmacodynamic or pharmacokinetic characteristics of the drug.

In specific embodiments, the co-suspension compositions described herein avoid coformulation effects associated with combination formulations that include three different active agent materials suspended within a single formulation. In particular embodiments, the co-suspension compositions described herein have been found to exhibit a lack of coformulation effects, even where each of the different active agents to be delivered is included in the suspension composition at widely ranging concentrations (e.g., to facilitate simultaneous delivery of different doses of each of active agent upon actuation of a metered dose inhaler). Embodiments of the compositions described herein avoid coformulation effects for each of the active agents contained therein. In certain such embodiments, the compositions described herein provide fine particle fraction (“FPF”), fine particle mass (“FPM”), delivered dose uniformity (“DDU”), area under the curve (“AUC₀₋₁₂”), and maximum plasma concentration (“Cₘₐₓ”) characteristics that do not deviate from those achieved by a comparable formulation wherein only one or two of the selected active agents are included.

Lack of a coformulation affect can be assessed in vivo or in vitro. The lack of a coformulation effect may be evidenced for a selected active agent where one or more pharmacokinetic characteristics of the active agent delivered from a combination formulation do not deviate from those achieved when the active agent is formulated as a single active agent at the same dose and delivered via the same route of administration using a comparable formulation. In addition or alternatively, in the present context, the lack of a coformulation effect may be evidenced for a selected active agent where one or more of the physical stability, chemical stability, and aerosol properties of a suspension formulation containing the active agent in combination with one or more additional active agents do not deviate from those achieved when the active agent is formulated as a single active agent at the same dose and delivered via the same route of administration using a comparable formulation.

As used herein, the phrases “do not deviate” or “does not deviate” signify that, for a given parameter, the performance achieved by a combination formulation is ±20% of that achieved by a comparable formulation including only one of the active agents included in the combination formulation. In certain embodiments, the performance achieved by a combination formulation does not vary from that achieved by a comparable formulation including only one of the active agents included in the combination. For example, a co-suspension as described herein, including three or more active agents, is considered to exhibit no coformulation effect for a given performance parameter (e.g., FPF, FPM, DDU, AUC₀₋₁₂, Cₘₐₓ, chemical stability, physical stability, and/or dose proportionality) when the performance achieved by the combination co-suspension for the selected parameter is within ±20% of that achieved by a comparable formulation including only a single active agent. In some embodiments, a co-suspension including three or more active agents is considered to exhibit no coformulation effect for a given performance parameter when the performance achieved by the combination co-suspension for the selected parameter is within ±15% of that achieved by a comparable formulation including only a single active agent. In yet other embodiments, a co-suspension including three or more active agents is considered to exhibit no coformulation effect for a given performance parameter when the performance achieved by the combination co-suspension for the selected parameter is within ±10% of that achieved by a comparable formulation including only a single active agent. In certain embodiments, with respect to each active agent at a given dose, the combination co-suspension compositions described exhibit no statistically significant difference to comparable formulations including only one of the active agents included in the combination in one or more of FPF, FPM, DDU, AUC₀₋₁₂, Cₘₐₓ, chemical stability, physical stability, and/or dose proportionality.

In specific embodiments, the methods described herein include methods for treating a pulmonary disease or disorder amenable to treatment by respiratory delivery of a co-suspension composition as described herein. For example, the compositions, methods and systems described herein may be used to treat inflammatory or obstructive pulmonary diseases or conditions. In certain embodiments, the compositions, methods and systems described herein may be used to treat patients suffering from a disease or disorder selected from asthma, COPD, exacerbation of airways hyper reactivity consequent to other drug therapy, allergic rhinitis, sinusitis, pulmonary vasoconstriction, inflammation, allergies, impeded respiration, respiratory distress syndrome, pulmonary hypertension, pulmonary vasoconstriction, and any other respiratory disease, condition, trait, genotype or phenotype that can respond to the administration of, for example, a LAMA, LABA, ICS, or other active agent as described herein, whether alone or in combination with other therapies. In certain embodiments, the compositions, systems and methods described herein may be used to treat pulmonary inflammation and obstruction associated with cystic fibrosis.

It will be readily understood that the embodiments, as generally described herein, are exemplary. The following more detailed description of various embodiments is not intended to limit the scope of the present disclosure, but is merely representative of various embodiments. As such, the specifics recited herein may include independently patentable subject matter. Moreover, the order of the steps or actions of the methods described in connection with the embodiments disclosed herein may be changed by those skilled in the art without departing from the scope of the present disclosure. In other words, unless a specific order of steps or actions is required for proper operation of the embodiment, the order or use of specific steps or actions may be modified.

### I. DEFINITIONS

Unless specifically defined otherwise, the technical terms, as used herein, have their normal meaning as understood in the art. The following terms are specifically defined for the sake of clarity.

The term “active agent” is used herein to include any agent, drug, compound, composition or other substance that may be used on, or administered to a human or animal for any purpose, including therapeutic, pharmaceutical, pharmacological, diagnostic, cosmetic and prophylactic agents and immunomodulators. The term “active agent” may be used interchangeably with the terms, “drug,” “pharmaceutical,” “medicament,” “drug substance,” or “therapeutic.” As used herein the “active agent” may also encompass natural or homeopathic products that are not generally considered therapeutic.

As used herein, the term “asthma” refers to asthma of whatever type or genesis, including intrinsic (non-allergic) asthma and extrinsic (allergic) asthma, mild asthma, moderate asthma, severe asthma, bronchitic asthma, exercise-induced asthma, occupational asthma and asthma induced following bacterial infection. Asthma is also to be understood as embracing wheezy-infant syndrome.

The terms “associate,” “associate with” or “association” refers to an interaction or relationship between a chemical entity, composition, or structure in a condition of proximity to a surface, such as the surface of another chemical entity, composition, or structure. The association includes, for example, adhesion, electrostatic attraction, Lifshitz-van der Waals interactions, and polar interactions. As used herein, “adhere” or “adhesion” is a form of association and is used as a generic term for all forces tending to cause a particle or mass to be attracted to a surface. “Adhere” also refers to bringing and keeping particles in contact with each other, such that there is substantially no visible separation between particles due to their different buoyancies in a propellant under normal conditions. In one embodiment, a particle that attaches to or binds to a surface is encompassed by the term “adhere.” Normal conditions may include storage at room temperature or under an accelerative force due to gravity. As described herein, active agent particles may associate with suspending particles to form a co-suspension, where there is substantially no visible separation between the suspending particles and the active agent particles or flocculates thereof due to differences in buoyancy within a propellant.

As used herein “AUC₀₋₁₂” refers to the area under the plasma concentration versus time curve (“AUC”) through the first 12 hours post administration. AUC₀₋₁₂ is widely used in the art as a measure of drug exposure. Measures of AUC, including AUC₀₋₁₂, are an accepted parameter for comparison of drug products, such as in bioequivalency and/or bioavailability studies.

The terms “chemically stable” and “chemical stability” refer to co-suspension formulations wherein the individual degradation products of active agent remain below the limits specified by regulatory requirements during the shelf life of the product for human use (e.g., 1% of total chromatographic peak area per ICH guidance Q3B(R2)) and there is acceptable mass balance (e.g., as defined in ICH guidance Q1E) between active agent assay and total degradation products.

The term “Cₘₐₓ” refers to the maximum or peak plasma concentration of a selected active agent post administration. Cₘₐₓ is widely used in the art as a measure of drug exposure and drug product comparability. For example, Cₘₐₓ is a standard parameter measured when comparing bioavailablity of an active agent and bioequivalence of different drug products.

As used herein, the terms “COPD” and “chronic obstructive pulmonary disease” encompass chronic obstructive lung disease (“COLD”), chronic obstructive airway disease (“COAD”), chronic airflow limitation (“CAL”) and chronic obstructive respiratory disease (“CORD”) and include chronic bronchitis, bronchiectasis, and emphysema.

The term “co-suspension” refers to a suspension of two or more types of particles having different compositions within a suspension medium, wherein one type of particle associates at least partially with one or more of the other particle types. The association leads to an observable change in one or more characteristics of at least one of the individual particle types suspended in the suspension medium. Characteristics modified by the association may include, for example, one or more of the rate of aggregation or flocculation, the rate and nature of separation, i.e. sedimentation or creaming, density of a cream or sediment layer, adhesion to container walls, adhesion to valve components, and rate and the level of dispersion upon agitation.

In the context of a composition containing or providing respirable aggregates, particles, drops, etc., such as compositions described herein, the term “fine particle mass” or “FPM” refers to the dose, either in total mass or fraction of the nominal dose or metered dose, that is within a respirable range. The dose that is within the respirable range is measured in vitro to be the dose that deposits beyond the throat stage of a cascade impactor, i.e., the sum of dose delivered at stages 3 through filter in a Next Generation Impactor operated at a flow rate of 30 l/min.

In the context of a composition containing or providing respirable aggregates, particles, drops, etc., such as compositions described herein, the term “fine particle fraction” or “FPF” refers to the proportion of the delivered material relative to the delivered dose (i.e., the amount that exits the actuator of a delivery device, such as an MDI) that is within a respirable range. The amount of delivered material within the respirable range is measured in vitro as the amount of material that deposits beyond the throat stage of a cascade impactor, e.g., the sum of the material delivered at stages 3 through filter in a Next Generation Impactor operated at a flow rate of 30 l/min.

As used herein, the term “inhibit” refers to a measurable lessening of the tendency of a phenomenon, symptom or condition to occur or the degree to which that phenomenon, symptom or condition occurs. The term “inhibit” or any form thereof, is used in its broadest sense and includes minimize, prevent, reduce, repress, suppress, curb, constrain, restrict, slow progress of and the like.

“Mass median aerodynamic diameter” or “MMAD” as used herein refers to the aerodynamic diameter of an aerosol below which 50% of the mass of the aerosol consists of particles with an aerodynamic diameter smaller than the MMAD, with the MMAD being calculated according to monograph 601 of the United States Pharmacopeia (“USP”).

When referred to herein, the term “optical diameter” indicates the size of a particle as measured by the Fraunhofer diffraction mode using a laser diffraction particle size analyzer equipped with a dry powder dispenser (e.g., Sympatec GmbH, Clausthal-Zellerfeld, Germany).

The term solution mediated transformation refers to the phenomenon in which a more soluble form of a solid material (i.e., particles with small radius of curvature (a driving force for Ostwald ripening), or amorphous material) dissolves and recrystallizes into the more stable crystal form that can coexist in equilibrium with its saturated propellant solution.

A “patient” refers to an animal in which a combination of active agents as described herein will have a therapeutic effect. In one embodiment, the patient is a human being.

“Perforated microstructures” refer to suspending particles that include a structural matrix that exhibits, defines or comprises voids, pores, defects, hollows, spaces, interstitial spaces, apertures, perforations or holes that allow the surrounding suspension medium to permeate, fill or pervade the microstructure, such as those materials and preparations described in U.S. Pat. No. 6,309,623 to Weers, et al. The primary form of the perforated microstructure is, generally, not essential, and any overall configuration that provides the desired formulation characteristics is contemplated herein. Accordingly, in one embodiment, the perforated microstructures may comprise approximately spherical shapes, such as hollow, suspending, spray-dried microspheres. However, collapsed, corrugated, deformed or fractured particulates of any primary form or aspect ratio may also be compatible.

As is true of suspending particles described herein, perforated microstructures may be formed of any biocompatible material that does not substantially degrade or dissolve in the selected suspension medium. While a wide variety of materials may be used to form the particles, in some embodiments, the structural matrix is associated with, or includes, a surfactant such as, a phospholipid or fluorinated surfactant. Although not required, the incorporation of a compatible surfactant in the perforated microstructure or, more generally, the suspending particles, may improve the stability of the respiratory dispersions, increase pulmonary deposition and facilitate the preparation of the suspension.

When used to refer to co-suspension compositions described herein, the terms “physical stability” and “physically stable” refer to a composition that is resistant to one or more of aggregation, flocculation, and particle size changes due to solution mediated transformations and is capable of substantially maintaining the MMAD of suspending particles and the fine particle mass. In one embodiment, physical stability may be evaluated through subjecting compositions to accelerated degradation conditions, such as by temperature cycling as described herein.

The term “respirable” generally refers to particles, aggregates, drops, etc. sized such that they can be inhaled and reach the airways of the lung.

The term “substantially insoluble” means that a composition is either totally insoluble in a particular solvent or it is poorly soluble in that particular solvent. The term “substantially insoluble” means that a particular solute has a solubility of less than one part per 100 parts solvent. The term “substantially insoluble” includes the definitions of “slightly soluble” (from 100 to 1000 parts solvent per 1 part solute), “very slightly soluble” (from 1000 to 10,000 parts solvent per 1 part solute) and “practically insoluble” (more than 10,000 parts solvent per 1 part solute) as given in Table 16-1 of Remington: The Science and Practice of Pharmacy, 21st ed. Lippincott, Williams & Wilkins, 2006, p. 212.

The term “surfactant,” as used herein, refers to any agent that preferentially adsorbs to an interface between two immiscible phases, such as the interface between water and an organic polymer solution, a water/air interface or organic solvent/air interface. Surfactants generally possess a hydrophilic moiety and a lipophilic moiety, such that, upon adsorbing to microparticles, they tend to present moieties to the continuous phase that do not attract similarly-coated particles, thus reducing particle agglomeration.

“Suspending particles” refer to a material or combination of materials that is acceptable for respiratory delivery, and acts as a vehicle for active agent particles. Suspending particles interact with the active agent particles to facilitate repeatable dosing, delivery or transport of active agent to the target site of delivery, i.e., the respiratory tract. The suspending particles described herein are dispersed within a suspension medium including a propellant or propellant system, and can be configured according to any shape, size or surface characteristic suited to achieving a desired suspension stability or active agent delivery performance. Exemplary suspending particles include particles that exhibit a particle size that facilitates respiratory delivery of active agent and have physical configurations suited to formulation and delivery of the stabilized suspensions as described herein.

The term “suspension medium” as used herein refers to a substance providing a continuous phase within which active agent particles and suspending particles can be dispersed to provide a co-suspension formulation. The suspension medium used in co-suspension formulations described herein includes propellant. As used herein, the term “propellant” refers to one or more pharmacologically inert substances which exert a sufficiently high vapor pressure at normal room temperature to propel a medicament from the canister of an MDI to a patient on actuation of the MDI's metering valve. Therefore, the term “propellant” refers to both a single propellant and to a combination of two or more different propellants forming a “propellant system.”

The terms “suspension stability” and “stable suspension” refer to suspension formulations capable of maintaining the properties of a co-suspension of active agent particles and suspending particles over a period of time. In one embodiment, suspension stability may be measured through delivered dose uniformity achieved by co-suspension compositions described herein.

A “therapeutically effective amount” is the amount of compound which achieves a therapeutic effect by inhibiting a disease or disorder in a patient or by prophylactically inhibiting or preventing the onset of a disease or disorder. A therapeutically effective amount may be an amount which relieves to some extent one or more symptoms of a disease or disorder in a patient; returns to normal either partially or completely one or more physiological or biochemical parameters associated with or causative of the disease or disorder; and/or reduces the likelihood of the onset of the disease of disorder.

### II. COMPOSITIONS

As active agents are combined into a fixed combination contained within a single formulation, the nature of the different active agents can result in coformulation effects that lead to formulation, stability, and deliverability challenges. Relative to a formulation including only a single active agent, the combination of multiple active agents within a single formulation may result in undesirable changes to one or more of the following: (i) the physical or chemical stability of the formulation components, including one or more of the actives; (ii) the deliverability or bioavailabilty of one or more of the actives; (iii) the metabolism of one or more of the actives; and (iv) the pharmacokinetic profile of one or more of the actives. The potential for undesirable coformulation effects is unpredictable, and the potential for coformulation challenges increases as the number of active agents combined increases, where different classes of active agents are combined, and where the doses of the combined active agents to be delivered from the combination formulation exhibit significant differences. Moreover, in the context of respiratory delivery of low-dose, potent active agents, achieving viable combination formulations can be particularly challenging. Coformulation effects that result in even small changes to drug availability or stability or to one or more characteristics of an aerosol generated for inhalation can have profound impacts on the therapeutic performance of an inhaled product.

The compositions described herein are co-suspensions that include three or more active agents co-suspended with suspending particles within a suspension medium. The active agents are provided as active agent particles, and the suspending particles are formed separately from and are different than the active particles. In particular embodiments, each of the three or more active agents is provided as a separate particulate constituent or species. In such an embodiment, the co-suspension includes a first species of active agent particle, a second species of active agent particle, a third species of active agent particle, and suspending particles all formed separately from one another and co-suspended within the suspension medium. In such embodiments, the three or more active agents may be provided as three different particle species, with the first species of active agent particles including a long-acting β₂ adrenergic receptor agonist (“LABA”), a second species of active agent particles including a including long-acting muscarinic antagonists (“LAMA”), and a third species of active agent particles including an inhaled corticosteroid (“ICS”). Of course, if desired, the compositions described herein may include one or more additional constituents. Moreover, variations and combinations of components of the compositions described herein may be used.

When delivered from an MDI, compositions described herein eliminate or substantially avoid coformulation effects often experienced with formulations including multiple active agents. For example, as exemplified by specific embodiments detailed herein, even where multiple classes of active agents are combined, and the delivered doses of the different active agents vary widely, the combination formulations described herein provide in-vitro and in-vivo delivery characteristics for each of the active agents that are comparable to the delivery characteristics of the same active agents when formulated and delivered individually.

Compositions described herein are suitable for delivery from an MDI, and embodiments of the compositions described herein include a LAMA active agent, a LABA active agent, and an ICS active agent. In such embodiments, the delivered dose of the active agents may be highly variable. As used herein in reference to relative delivered doses of active agents, the terms “highly variable,” “vary widely,” and “significant difference” refer to a delivered dose of a first active agent that is at least five fold higher than the delivered dose of another active agent coformulated as a fixed combination. ICS active agents are often administered at significantly higher doses than LAMA and LABA active agents, and in specific embodiments, the compositions described herein may be formulated to provide a delivered dose of ICS that is at least five times greater than the delivered dose of LAMA active agent (i.e., the ratio of the delivered dose of ICS to the delivered dose of LAMA per actuation of an MDI is greater than or equal to 5). In other embodiments, the compositions described herein may be formulated to provide a delivered dose of ICS that is at least five times greater than the delivered dose of LABA active agent (i.e., the ratio of the delivered dose of ICS to the delivered dose of LABA per actuation of an MDI is greater than or equal to 5). In still further embodiments, the compositions described herein may be formulated to provide a delivered dose of ICS that is at least five times greater than both the delivered dose of LABA active agent and the delivered does of LAMA active agent.

The compositions described herein exhibit desirable dose proportionality, FPF, FPM, and DDU characteristics even when formulated to provide a fixed combination of LAMA, LABA, and ICS active agents delivered at highly variable doses. For example, embodiments of the compositions described herein can achieve a DDU of ±30%, or better for each of the three or more active agents included therein. In one such embodiment, compositions described herein achieve a DDU of ±25%, or better, for each of the three or more active agents included therein. In another such embodiment, compositions described herein achieve a DDU of ±20%, or better, for each of the three or more active agents included therein. Moreover, co-suspension compositions according to the present description serve to substantially preserve FPF and FPM performance throughout emptying of an MDI canister, even after being subjected to accelerated degradation conditions. For instance, compositions according to the present description maintain as much as 80%, 90%, 95%, or more, of the original FPF or FPM performance, even after being subjected to accelerated degradation conditions.

In compositions according to the present description, the active agent particles exhibit an association with the suspending particles such that the active agent particles and suspending particles co-locate within the suspension medium. Generally, due to density differences between distinct species of particles and the medium within which they are suspended (e.g., a propellant or propellant system), buoyancy forces cause creaming of particles with lower density than the propellant and sedimentation of particles with higher density than the propellant. Therefore, in suspensions that consist of a mixture of different types of particles with different density or different tendencies to flocculate, sedimentation or creaming behavior is expected to be specific to each of the different particle types and expected to lead to separation of the different particle types within the suspension medium. The combinations of propellant, active agent particles, and suspending particles described herein provide co-suspensions including combinations of three or more active agents wherein the active agent particles and suspending particles co-locate within the propellant (i.e., the active agent particles associate with the suspending particles such that suspending particles and active agent particles do not exhibit substantial separation relative to each other, such as by differential sedimentation or creaming, even after a time sufficient for the formation of a cream or sediment layer).

The combination co-suspensions of active agent particles and suspending particles according to the present description provide desirable chemical stability, suspension stability and active agent delivery characteristics. For example, in certain embodiments, when present within an MDI canister, co-suspensions as described herein can inhibit one or more of the following: differential sedimentation or creaming of active agent particles and suspending particles; solution mediated transformation of active agent material; chemical degradation of a component of the formulation, including of active agent material; and loss of active agent to the surfaces of the container closure system, in particular the metering valve components. Such qualities work to achieve and preserve aerosol performance as the composition is delivered from an MDI such that desirable FPF, FPM, and DDU characteristics are achieved and substantially maintained throughout emptying of an MDI canister within which the co-suspension formulation is contained. Additionally, co-suspensions according to the present description can provide a physically and chemically stable formulation that provides consistent dosing characteristics for three or more active agents, even where such active agents are delivered at significantly different doses, while utilizing an HFA suspension medium that does not require modification by the addition of, for example, cosolvents, antisolvents, solubilizing agents or adjuvants.

Co-suspension compositions described herein provide the added benefit of achieving such performance while being formulated using non-CFC propellants. In specific embodiments, the compositions described herein achieve one or more of a targeted DDU, FPF or FPM, while being formulated with suspension medium including only one or more non-CFC propellants and without the need to modify the characteristics of the non-CFC propellant, such as by the addition of, for example, one or more cosolvent, antisolvent, solubilizing agent, adjuvant or other propellant modifying material.

(i) Suspension Medium

The suspension medium included in a composition described herein includes one or more propellants. In general, suitable propellants for use as suspension mediums are those propellant gases that can be liquefied under pressure at room temperature, and upon inhalation or topical use, are safe and toxicologically innocuous. Additionally, it is desirable that the selected propellant be relatively non-reactive with the suspending particles and active agent particles. Exemplary compatible propellants include hydrofluoroalkanes (HFAs), perfluorinated compounds (PFCs), and chlorofluorocarbons (CFCs).

Specific examples of propellants that may be used to form the suspension medium of the co-suspensions disclosed herein include 1,1,1,2-tetrafluoroethane (CF₃CH₂F) (HFA-134a), 1,1,1,2,3,3,3-heptafluoro-n-propane (CF₃CHFCF₃) (HFA-227), perfluoroethane, monochloro-fluoromethane, 1,1 difluoroethane, and combinations thereof. Even further, suitable propellants include, for example: short chain hydrocarbons; C₁₋₄ hydrogen-containing chlorofluorocarbons such as CH₂ClF, CCl₂FCHClF, CF₃CHClF, CHF₂CClF₂, CHClFCHF₂, CF₃CH₂Cl, and CClF₂CH₃; C₁₋₄ hydrogen-containing fluorocarbons (e.g., HFAs) such as CHF₂CHF₂, CF₃CH₂F, CHF₂CH₃, and CF₃CHFCF₃; and periluorocarbons such as CF₃CF₃ and CF₃CF₂CF₃.

Specific fluorocarbons, or classes of fluorinated compounds, that may be used as suspension media include, but are not limited to, fluoroheptane, fluorocycloheptane, fluoromethylcycloheptane, fluorohexane, fluorocyclohexane, fluoropentane, fluorocyclopentane, fluoromethylcyclopentane, fluorodimethyl-cyclopentanes, fluoromethylcyclobutane, fluorodimethylcyclobutane, fluorotrimethyl-cyclobutane, fluorobutane, fluorocyclobutane, fluoropropane, fluoroethers, fluoropolyethers and fluorotriethylamines. These compounds may be used alone or in combination with more volatile propellants.

In addition to the aforementioned fluorocarbons and hydrofluoroalkanes, various exemplary chlorofluorocarbons and substituted fluorinated compounds may also be used as suspension media. In this respect, FC-11 (CCl₃F), FC-11B1 (CBrCl₂F), FC-11B2 (CBr₂ClF), FC12B2 (CF₂Br₂), FC21 (CHCl₂F), FC21B1 (CHBrClF), FC-21B2 (CHBr₂F), FC-31B1 (CH₂BrF), FC113A (CCl₃CF₃), FC-122 (CClF₂CHCl₂), FC-123 (CF₃CHCl₂), FC-132 (CHClFCHClF), FC-133 (CHClFCHF₂), FC-141 (CH₂ClCHClF), FC-141B (CCl₂FCH₃), FC-142 (CHF₂CH₂Cl), FC-151 (CH₂FCH₂Cl), FC-152 (CH₂FCH₂F), FC-1112 (CClF═CClF), FC-1121 (CHCl═CFCl) and FC-1131 (CHCl═CHF) may also be used, while recognizing the possible attendant environmental concerns. As such, each of these compounds may be used, alone or in combination with other compounds (i.e., less volatile fluorocarbons) to form the stabilized suspensions disclosed herein.

The suspension medium may be formed of a single propellant. In other embodiments, a combination of propellants may be used to form the suspension medium. In some embodiments, relatively volatile compounds may be mixed with lower vapor pressure components to provide suspension media having specified physical characteristics selected to improve stability or enhance the deliverability and/or bioavailability of the dispersed active agents. In some embodiments, the lower vapor pressure compounds will comprise fluorinated compounds (e.g. fluorocarbons) having a boiling point greater than about 25° C. In some embodiments, lower vapor pressure fluorinated compounds for use in the suspension medium may include perfluorooctylbromide C₈F₁₇Br (PFOB or perflubron), dichlorofluorooctane C₈F₁₆Cl₂, perfluorooctylethane C₈F₁₇C₂H₅ (PFOE), perfluorodecylbromide C₁₀F₂₁Br (PFDB) or perfluorobutylethane C₄F₉C₂H₅. In certain embodiments, these lower vapor pressure compounds are present in a relatively low level. Such compounds may be added directly to the suspension medium or may be associated with the suspending particles.

The suspension medium included in compositions as described herein may be formed of a propellant or propellant system that is substantially free of additional materials, including, for example, antisolvents, solubilizing agents, cosolvents or adjuvants. For example, the suspension medium may be formed of a non-CFC propellant or propellant system, such as an HFA propellant or propellant system that is substantially free of additional materials. Such embodiments simplify the formulation and manufacture of pharmaceutical compositions suited for respiratory delivery of the multiple active agents included in the co-suspension compositions.

(ii) Active Agent Particles

The active agent particles included in the co-suspensions described herein are respirable particles formed of a material capable of being dispersed and suspended within the suspension medium and are sized to facilitate delivery of respirable particles from the co-suspension. In one embodiment, therefore, the active agent particles are provided as a micronized material wherein at least 90% of the active agent particles by volume exhibit an optical diameter of about 7 μm or less. In other embodiments, the active agent particles are provided as a micronized material wherein at least 90% of the active agent particles by volume exhibit an optical diameter selected from a range of about 7 μm to about 1 μm, about 5 μm to about 2 μm, and about 3 μm to about 2 μm. In other embodiments, the active agent particles are provided as a micronized material wherein at least 90% of the active agent particles by volume exhibit an optical diameter selected from 6 μm or less, 5 μm or less, 4 μm or less, or 3 μm or less. In another embodiment, the active agent particles are provided as a micronized material wherein at least 50% of the active agent particle material by volume exhibits an optical diameter of about 4 μm or less. In further embodiments, the active agent particles are provided as a micronized material wherein at least 50% of the active agent particle material by volume exhibits an optical diameter selected from about 3 μm or less, about 2 μm or less, about 1.5 μm or less, and about 1 μm or less. In still further embodiments, the active agent particles are provided as a micronized material wherein at least 50% of the active agent particles by volume exhibit an optical diameter selected from a range of about 4 μm to about 1 μm, about 3 μm to about 1 μm, about 2 μm to about 1 μm, about 1.3 μm, and about 1.9 μm.

In specific embodiments, each of the different species of active agent particles are formed of active agent material that is entirely or substantially crystalline, i.e., a majority of the active agent molecules are arranged in a regularly repeating pattern, over a long range of external face planes. In another embodiment, one or more of the different species of active agent particles may include an active agent present in both crystal and amorphous states. In yet another embodiment, one or more of the different species active agent particles may include an active agent present in substantially an amorphous state, i.e., the active agent molecules are overall noncrystalline in nature and do not have a regularly repeating arrangement maintained over a long range. The active agents included in the compositions described herein are substantially insoluble in the suspension medium. In particular embodiments, for example, each of the active agents are substantially insoluble in the suspension medium, with one or more of such active agents being very slightly soluble in the suspension medium. In further embodiments, each of the active agents are substantially insoluble in the suspension medium, with one or more of such active agents being practically insoluble in the suspension medium.

Any suitable process may be employed to achieve micronized active agent material for use as or inclusion in active agent particles described herein. Such processes include, but are not limited to, micronization by milling or grinding processes, crystallization or recrystallization processes, and processes using precipitation from supercritical or near-supercritical solvents, spray drying, spray freeze-drying, or lyophilization. Patent references teaching suitable methods for obtaining micronized active agent particles are described, for example, in U.S. Pat. No. 6,063,138, U.S. Pat. No. 5,858,410, U.S. Pat. No. 5,851,453, U.S. Pat. No. 5,833,891, U.S. Pat. No. 5,707,634, and International Patent Publication No. WO 2007/009164.

A variety of therapeutic or prophylactic agents can be utilized as active agents in the compositions disclosed herein. Exemplary active agents include those that may be administered in the form of aerosolized medicaments, and active agents suitable for use in the compositions described herein include those that may be presented in a form or formulated in a manner which is dispersible within the selected suspension medium (e.g., is substantially insoluble or exhibits a solubility in the suspension medium that substantially maintains a co-suspension formulation), is capable of forming a co-suspension with the suspending particles, and is subject to respirable uptake in physiologically effective amounts. The active agents that may be utilized in forming the active agent particles described herein can have a variety of biological activities.

Examples of specific active agents that may be included in a composition according to the present description may for example, short-acting beta agonists, e.g., bitolterol, carbuterol, fenoterol, hexoprenaline, isoprenaline (isoproterenol), levosalbutamol, orciprenaline (metaproterenol), pirbuterol, procaterol, rimiterol, salbutamol (albuterol), terbutaline, tulobuterol, reproterol, ipratropium and epinephrine; long-acting β₂ adrenergic receptor agonist (LABA), e.g., bambuterol, clenbuterol, formoterol, salmeterol; ultra long-acting β₂ adrenergic receptor agonists, e.g., carmoterol, milveterol, indacaterol, and saligenin- or indole-containing and adamantyl-derived β₂ agonists; corticosteroids, e.g., beclomethasone, budesonide, ciclesonide, flunisolide, fluticasone, methyl-prednisolone, mometasone, prednisone and trimacinolone; anti-inflammatories, e.g. fluticasone propionate, beclomethasone dipropionate, flunisolide, budesonide, tripedane, cortisone, prednisone, prednisilone, dexamethasone, betamethasone, or triamcinolone acetonide; antitussives, e.g., noscapine; bronchodilators, e.g., ephedrine, adrenaline, fenoterol, formoterol, isoprenaline, metaproterenol, salbutamol, albuterol, salmeterol, terbutaline; muscarinic antagonists, including long-acting muscarinic antagonists (LAMA), e.g., glycopyrronium, dexipirronium, scopolamine, tropicamide, pirenzepine, dimenhydrinate, tiotropium, darotropium, aclidinium, trospium, ipatropium, atropine, benzatropin, or oxitropium; and anti-infectives.

Where appropriate, the active agents provided in the composition, including but not limited to those specifically described herein, may be used in the form of salts (e.g., alkali metal or amine salts or as acid addition salts) or as esters, solvates (hydrates), derivatives, or a free base. Additionally, the active agents may be in any crystalline form or isomeric form or mixture of isomeric forms, for example, as pure enantiomers, a mixture of enantiomers, as racemates or as mixtures thereof. In this regard, the form of the active agents may be selected to optimize the activity and/or stability of the active agent and/or to minimize the solubility of the active agent in the suspension medium.

The compositions described herein include a LABA active agent in combination with a LAMA active agent and an ICS active agent. The LABA active agent can be selected from, for example, bambuterol, clenbuterol, formoterol, salmeterol, carmoterol, milveterol, indacaterol, and saligenin- or indole-containing and adamantyl-derived β₂ agonists, and any pharmaceutically acceptable salts, esters, isomers or solvates thereof. In certain such embodiments, the active agent is selected from formoterol and its pharmaceutically acceptable salts, esters, isomers or solvates thereof.

Formoterol can be used to treat inflammatory or obstructive pulmonary diseases and disorders such as, for example, those described herein. Formoterol has the chemical name (±)-2-hydroxy-5-[(1RS)-1-hydroxy-2-[[(1RS)-2-(4-methoxyphenyl)-1-methylethyl]-amino]ethyl] formanilide, and is commonly used in pharmaceutical compositions as the racemic fumarate dihydrate salt. Where appropriate, formoterol may be used in the form of salts (e.g. alkali metal or amine salts or as acid addition salts) or as esters or as solvates (hydrates). Additionally, the formoterol may be in any crystalline form or isomeric form or mixture of isomeric forms, for example a pure enantiomer, a mixture of enantiomers, a racemate or a mixture thereof. In this regard, the form of formoterol may be selected to optimize the activity and/or stability of formoterol and/or to minimize the solubility of formoterol in the suspension medium. Pharmaceutically acceptable salts of formoterol include, for example, salts of inorganic acids such as hydrochloric, hydrobromic, sulfuric and phosphoric acids, and organic acids such as fumaric, maleic, acetic, lactic, citric, tartaric, ascorbic, succinic, glutaric, gluconic, tricarballylic, oleic, benzoic, p-methoxybenzoic, salicylic, o- and p-hydroxybenzoic, p-chlorobenzoic, methanesulfonic, p-toluenesulfonic and 3-hydroxy-2-naphthalene carboxylic acids. Hydrates of formoterol are described, for example, in U.S. Pat. No. 3,994,974 and U.S. Pat. No. 5,684,199. Specific crystalline forms are described, for example, in WO95/05805, and specific isomers of formoterol are described in U.S. Pat. No. 6,040,344.

In specific embodiments, the formoterol material utilized to form the formoterol particles is formoterol fumarate, and in one such embodiment, the formoterol fumarate is present in the dihydrate form. Where the compositions described herein include formoterol, in certain embodiments, the compositions described herein may include formoterol at a concentration that achieves a delivered dose selected from between about 0.1 μg and about 30 μg, 0.1 μg and about 1 μg, about 1 μg and about 10 μg, about 2 μg and 5 μg, about 2 μg and about 10 μg, about 5 μg and about 10 μg, and 3 μg and about 30 μg per actuation of an MDI. In other embodiments, the compositions described herein may include formoterol in an amount sufficient to provide a delivered dose selected from up to about 30 μg, up to about 10 μg, up to about 5 μg, up to about 2.5 μg, up to about 2 μg, or up to about 1.5 μg per actuation of an MDI.

The compositions described herein include a long-acting muscarinic antagonist (LAMA) active agent. Examples of LAMA active agents that may be used in the compositions described herein include, for example, glycopyrronium, dexipirronium, tiotropium, trospium, aclidinium and darotropium, including any pharmaceutically acceptable salts, esters, isomers or solvates thereof. Glycopyrronium may be provided as a salt (e.g. alkali metal or amine salts, or as acid addition salts), esters or solvate (hydrates). Suitable counter ions of glycopyrronium include, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate and benzenesulfonate. In particular embodiments, the compositions described herein include the bromide salt of glycopyrronium, namely (3-[(cyclopentylhydroxyphenylacetyl)oxy]-1,1-dimethyl-, bromide). The bromide salt of glycopyrronium is commonly referred to as glycopyrrolate. Glycopyrrolate is commercially available and can be prepared according to the procedures set out in U.S. Pat. No. 2,956,062, the contents of which are incorporated herein by reference. The structure of glycopyrronium bromide is shown below:

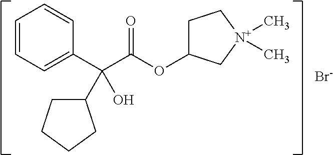

Where the compositions described herein include glycopyrrolate, in certain embodiments, the compositions may include sufficient glycopyrrolate to provide a delivered dose selected from between about 1 μg and about 100 μg, about 15 μg and about 100 μg, about 5 μg and about 80 μg, and about 2 μg and about 40 μg per actuation of an MDI. In other such embodiments, the formulations include sufficient glycopyrrolate to provide a delivered dose selected from up to about 100 μg, up to about 80 μg, up to about 40 μg, up to about 20 μg, up to about 10 μg per actuation, up to about 5 μg of an MDI. In yet further embodiments, the formulations include sufficient glycopyrrolate to provide a delivered dose selected from about 2 μg, 5 μg, 9 μg, 18 μg, 36 μg and 72 μg per actuation of the MDI.

The compositions described herein include an ICS. The ICS can be selected, for example, from beclomethasone, budesonide, ciclesonide, flunisolide, fluticasone, methyl-prednisolone, mometasone, prednisone and trimacinolone, including any pharmaceutically acceptable salts, esters, isomers or solvates thereof. In specific embodiments, the ICS active agent is selected from mometasone and budesonide.

Mometasone, pharmaceutically acceptable salts of mometasone, such as mometasone furoate, and preparation of such materials are known, and described, for example, in U.S. Pat. No. 4,472,393, U.S. Pat. No. 5,886,200, and U.S. Pat. No. 6,177,560. Mometasone is suitable for use in treating diseases or disorders associated with pulmonary inflammation or obstruction, such as those described herein (see, e.g., U.S. Pat. No. 5,889,015, U.S. Pat. No. 6,057,307, U.S. Pat. No. 6,057,581, U.S. Pat. No. 6,677,322, U.S. Pat. No. 6,677,323 and U.S. Pat. No. 6,365,581).

Where the compositions described herein include mometasone as an ICS, in particular embodiments, the compositions include a pharmaceutically acceptable salt, ester, isomer, or solvate of mometasone in an amount sufficient to provide a target delivered dose selected from between about 20 μg and about 400 μg, between about 20 μg and about 200 μg, between about 50 μg and about 200 μg, between about 100 μg and about 200 μg, between about 20 μg and about 100 μg, and between about 50 μg and about 100 μg per actuation of an MDI. In still other embodiments, the compositions described herein may include mometasone, including any pharmaceutically acceptable salts, esters, isomers or solvates thereof, in an amount sufficient to provide a targeted delivered dose selected from up to about 400 μg, up to about 300 up to about 200 μg, up to about 100 μg, up to about 200 μg, and up to about 25 μg per actuation of an MDI.

Budesonide is also well known and described in, for example, U.S. Pat. No. 3,929,768. In particular embodiments, compositions described herein may include any pharmaceutically acceptable salt, ester, isomer, or solvate of budesonide in an amount sufficient to provide target delivered dose selected from between about 5 μg and about 80 μg, between about 5 μg and about 40 μg, between about 5 μg and about 80 μg, between about 20 μg and about 40 μg, between about 40 μg and about 80 μg, between about 80 μg and about 160 μg, between about 80 μg and about 200 μg, and between about 100 μg and about 240 μg per actuation of an MDI. In still other embodiments, the compositions described herein may include budesonide, including any pharmaceutically acceptable salts, esters, isomers or solvates thereof, in an amount sufficient to provide a targeted delivered dose selected from up to about 20 μg, up to about 40 μg, up to about 80 μg, up to about 100 μg, up to about 160 μg, up to about 200 μg, and up to about 240 μg per actuation of an MDI.

Where budesonide is selected as the ICS, compositions according to the present description may be formulated to include a combination of formoterol as the LABA active agent, glycopyrronium as the LAMA active agent, and budesonide as the ICS active agent. In such embodiments, the composition can be formulated to provide a delivered dose of formoterol of up to about 10 μg per actuation, a delivered dose of glycopyrronium of up to about 40 μg per actuation, and a delivered dose of budesonide of up to about 240 μg per actuation. In another such embodiment, the composition can be formulated to provide a delivered dose of formoterol of up to about 10 μg per actuation, a delivered dose of glycopyrronium of up to about 20 μg per actuation, and a delivered dose of budesonide of up to about 160 μg per actuation. In another such embodiment, the composition can be formulated to provide a delivered dose of formoterol of up to about 10 μg per actuation, a delivered dose of glycopyrronium of up to about 20 μg per actuation, and a delivered dose of budesonide of up to about 80 μg per actuation. In yet another such embodiment, the composition can be formulated to provide a delivered dose of formoterol of up to about 5 μg per actuation, a delivered dose of glycopyrronium of up to about 40 μg per actuation, and a delivered dose of budesonide of up to about 240 μg per actuation. In still another such embodiment, the composition can be formulated to provide a delivered dose of formoterol of up to about 5 μg per actuation, a delivered dose of glycopyrronium of up to about 20 μg per actuation, and a delivered dose of budesonide of up to about 160 μg per actuation. In still another such embodiment, the composition can be formulated to provide a delivered dose of formoterol of up to about 5 μg per actuation, a delivered dose of glycopyrronium of up to about 10 μg per actuation, and a delivered dose of budesonide of up to about 80 μg per actuation. In another such embodiment, the composition can be formulated to provide a delivered dose of formoterol of up to about 2.5 μg per actuation, a delivered dose of glycopyrronium of up to about 10 μg per actuation, and a delivered dose of budesonide of up to about 160 μg per actuation. In yet another such embodiment, the composition can be formulated to provide a delivered dose of formoterol of up to about 2.5 μg per actuation, a delivered dose of glycopyrronium of up to about 10 μg per actuation, and a delivered dose of budesonide of up to about 80 μg per actuation. In still another such embodiment, the composition can be formulated to provide a delivered dose of formoterol of up to about 2.5 μg per actuation, a delivered dose of glycopyrronium of up to about 7.5 μg per actuation, and a delivered dose of budesonide of up to about 40 μg per actuation. In another such embodiment, the composition can be formulated to provide a delivered dose of formoterol of up to about 5.0 μg per actuation, a delivered dose of glycopyrronium of up to about 7.5 μg per actuation, and a delivered dose of budesonide of up to about 160 μg per actuation. In another such embodiment, the composition can be formulated to provide a delivered dose of formoterol of up to about 5.0 μg per actuation, a delivered dose of glycopyrronium of up to about 7.5 μg per actuation, and a delivered dose of budesonide of up to about 80 μg per actuation. In another such embodiment, the composition can be formulated to provide a delivered dose of formoterol of up to about 5.0 μg per actuation, a delivered dose of glycopyrronium of up to about 7.5 μg per actuation, and a delivered dose of budesonide of up to about 40 μg per actuation.

(iii) Suspending Particles

Though various forms of suspending particles may be used, the suspending particles are typically formed from a dry, particulate, and pharmacologically inert material that is acceptable for inhalation and is substantially insoluble in the propellant selected. In particular embodiments, the suspending particles are very slightly soluble in the suspension medium. In further embodiments, the suspending particles are practically insoluble in the suspension medium. Suspending particles suitable for use in the compositions described herein are prepared to exhibit a particle size distribution within a respirable range (i.e., respirable suspending particles). In particular embodiments, therefore, the MMAD of the suspending particles will not exceed about 10 μm but is not lower than about 500 nm. In an alternative embodiment, the MMAD of the suspending particles is between about 5 μm and about 750 nm. In yet another embodiment, the MMAD of the suspending particles is between about 1 μm and about 3 μm. When used in an embodiment for nasal delivery from an MDI, the MMAD of the suspending particles is between 10 μm and 50 μm.

In order to achieve respirable suspending particles within the MMAD ranges described, the suspending particles will typically exhibit a volume median optical diameter between about 0.2 μm and about 50 μm. In one embodiment, the suspending particles exhibit a volume median optical diameter that does not exceed about 25 μm. In another embodiment, the suspending particles exhibit a volume median optical diameter selected from between about 0.5 μm and about 15 μm, between about 1.5 μm and about 10 μm, and between about 2 μm and about 5 μm.

The relative amount of suspending particles to active agent particles is selected to achieve a co-suspension as contemplated herein. It has been found that, with compositions as disclosed herein including a combination of LABA, LAMA, and ICS active agents, the total mass of the suspending particles to the total mass of active agent particles may range from below 1:1 to well above 1:1. In specific embodiments, the ratio of the total mass of the suspending particles to the total mass of the active agent particles may be selected from between about 0.5:1 and about 75:1, between about 0.5:1 and about 50:1, between about 0.5:1 and about 35:1, between about 0.5:1 and about 25:1, between about 0.5:1 and about 15:1, between about 0.5:1 and about 10:1, and between about 0.5:1 and about 5:1. In further embodiments, the ratio of the total mass of the suspending particles to the total mass of the active agent particles may be selected from between about 1.5:1 and about 75:1, between about 1.5:1 and about 50:1, between about 1.5:1 and about 35:1, between about 1.5:1 and about 25:1, between about 1.5:1 and about 15:1, between about 1.5:1 and about 10:1, and between about 1.5:1 and about 5:1. In other embodiments, the ratio of the total mass of the suspending particles to the total mass of the active agent particles may be selected from between about 2.5:1 and about 75:1, between about 2.5:1 and about 50:1, between about 2.5:1 and about 35:1, between about 2.5:1 and about 25:1, between about 2.5:1 and about 15:1, between about 2.5:1 and about 10:1, and between about 2.5:1 and about 5:1. In yet further embodiments, the ratio of the total mass of the suspending particles to the total mass of the active agent particles may be selected from between about 5:1 and about 75:1, between about 5:1 and about 50:1, between about 5:1 and about 35:1, between about 5:1 and about 25:1, between about 5:1 and about 15:1, and between about 5:1 and about 10:1.

Phospholipids from both natural and synthetic sources may be used in preparing suspending particles suitable for use in the compositions described herein. In particular embodiments, the phospholipid chosen will have a gel to liquid crystal phase transition of greater than about 40° C. Exemplary phospholipids are relatively long chain (i.e., C₁₆-C₂₂) saturated lipids and may comprise saturated phospholipids, such as saturated phosphatidylcholines having acyl chain lengths of 16 C or 18 C (palmitoyl and stearoyl). Exemplary phospholipids include phosphoglycerides such as dipalmitoylphosphatidylcholine, disteroylphosphatidylcholine, diarachidoylphosphatidylcholine, dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, and long-chain saturated phosphatidylinositols. In specific embodiments, the suspending particles are formed using 1,2 distearoyl-sn-glycero-3-phosphocholine (DSPC) as a phospholipid material. In such embodiments, the DPSC suspending particles may additionally include calcium chloride (CaCl₂). Methods suitable for preparing suspending particles as described herein using DSPC are described, for example, in U.S. Pat. No. 8,324,266 and in Cosuspensions of microcrystals and engineered microparticles for uniform and efficient delivery of respiratory therapeutics from pressurized metered dose inhalers, Vehring, R, Lechuga-Ballesteros, D, Joshi, V, Noga, B, Dwivedi, S K: Langmuir 2012, 28(42):15015-23. Additional excipients are disclosed in International Patent Publication No. WO 96/32149 and U.S. Pat. Nos. 6,358,530, 6,372,258 and 6,518,239.

The suspending particles described herein, such as, for example, suspending particles formed using one or more phospholipids, can be formed to exhibit a desired surface rugosity (roughness), which can further reduce inter-particle interactions and improve aerosolization by reducing the surface area available for particle-particle interaction. In further embodiments, if suitable, a phospholipid that is naturally occurring in the lung may be used in forming the suspending particles.

In another aspect, the suspending particles utilized in the compositions described herein may be selected to increase storage stability of the selected active agent, similar to that disclosed in International Patent Publication No. WO 2005/000267. For example, in one embodiment, the suspending particles may include pharmaceutically acceptable glass stabilization excipients having a Tg of at least 55° C., at least 75° C., or at least 100° C. Glass formers suitable for use in compositions described herein include, but are not limited to, one or more of trileucine, sodium citrate, sodium phosphate, ascorbic acid, inulin, cyclodextrin, polyvinyl pyrrolidone, mannitol, sucrose, trehalose, lactose, and, proline. Examples of additional glass-forming excipients are disclosed in U.S. Pat. Nos. RE 37,872, 5,928,469, 6,258,341, and 6,309,671.

The suspending particles may be designed, sized and shaped as desired to provide desirable stability and active agent delivery characteristics. In one exemplary embodiment, the suspending particles comprise perforated microstructures as described herein. Where perforated microstructures are used as suspending particles in the compositions described herein, they may be formed using one or more excipients as described herein. For example, in particular embodiments, perforated microstructures may include at least one of the following: lipids, phospholipids, nonionic detergents, nonionic block copolymers, ionic surfactants, biocompatible fluorinated surfactants and combinations thereof, particularly those approved for pulmonary use. Specific surfactants that may be used in the preparation of perforated microstructures include poloxamer 188, poloxamer 407 and poloxamer 338. Other specific surfactants include oleic acid or its alkali salts. In one embodiment, the perforated microstructures include greater than about 10% w/w surfactant.

In some embodiments, suspending particles may be prepared by forming an oil-in-water emulsion, using afluorocarbon oil (e.g., perfluorooctyl bromide, perfluorodecalin) which may be emulsified using a surfactant such as a long chain saturated phospholipid. The resulting perfluorocarbon in water emulsion may be then processed using a high pressure homogenizer to reduce the oil droplet size. The perfluorocarbon emulsion may be fed into a spray dryer, optionally with an active agent solution, if it is desirable to include active agent within the matrix of the perforated microstructures. As is well known, spray drying is a one-step process that converts a liquid feed to a dried particulate form. Spray drying has been used to provide powdered pharmaceutical material for various administrative routes, including inhalation. Operating conditions of a spray dryer (such as inlet and outlet temperature, feed rate, atomization pressure, flow rate of the drying air and nozzle configuration) can be adjusted to produce the desired particle size and yield of the resulting dry, particulate microstructures to serve as suspending particles. Such methods of producing exemplary perforated microstructures are disclosed in, for example, U.S. Pat. No. 6,309,623 to Weers et al. Methods suitable for preparing suspending particles as described herein are also described in Cosuspensions of microcrystals and engineered microparticles for uniform and efficient delivery of respiratory therapeutics from pressurized metered dose inhalers, Vehring, R, Lechuga-Ballesteros, D, Joshi, V, Noga, B, Dwivedi, S K: Langmuir 2012, 28(42):15015-23.

Furthermore, suspending particles as described herein may include bulking agents, such as polymeric particles. Polymeric polymers may be formed from biocompatible and/or biodegradable polymers, copolymers or blends. In one embodiment, polymers capable of forming aerodynamically light particles may be used, such as functionalized polyester graft copolymers and biodegradable polyanhydrides. For example, bulk eroding polymers based on polyesters including poly(hydroxy acids) can be used. Polyglycolic acid (PGA), polyactic acid (PLA) or copolymers thereof may be used to form suspending particles. The polyester may include a charged or functionalizable group, such as an amino acid. For example, suspending particles may be formed of poly(D,L-lactic acid) and/or poly(D,L-lactic-co-glycolic acid) (PLGA), which incorporate a surfactant such as DPPC.

The pharmaceutical compositions described herein are suited for simultaneous, respiratory delivery of three or more active agents via an MDI. In particular embodiments, the compositions described herein provide simultaneous respiratory delivery of a LABA active agent, a LAMA active agent, and an ICS active agent via an MDI in a manner that achieves desirable DDU of each active agent included in a combination, even with highly variable target delivered doses for each of the three or more active agents. Even when delivering very low doses one or more active agents (e.g., one or both of a LAMA active agent and a LABA active agent) and relatively much higher doses of one or more of the other active agents included (e.g., an ICS active agent), compositions described herein can achieve a DDU of ±30%, or better, for each of the LAMA, LABA, and ICS active agents throughout emptying of an MDI canister. In one such embodiment, compositions described herein achieve a DDU of ±25%, or better, for each of the LAMA, LABA, and ICS active agents throughout emptying of an MDI canister. In yet another such embodiment, compositions described herein achieve a DDU for the active agent of ±20%, or better, for each of the LAMA, LABA, and ICS active agents throughout emptying of an MDI canister.

Pharmaceutical compositions described herein also serve to substantially preserve FPF and FPM performance throughout emptying of an MDI canister, even after being subjected to accelerated degradation conditions. For instance, compositions according to the present description maintain as much as 80%, 90%, 95%, or more, of the original FPF and FPM performance throughout emptying of an MDI canister, even after being subjected to accelerated degradation conditions. Compositions described herein may also achieve such performance while being formulated using non-CFC propellants and eliminating or substantially avoiding pharmaceutical effects often experienced with compositions incorporating three or more active agents. In specific embodiments, the compositions described herein achieve desired one or all of a targeted DDU, FPF and FPM performance for each of a LABA, a LAMA, and an ICS active agent, while being formulated with suspension medium including only one or more non-CFC propellants and without the need to modify the characteristics of the non-CFC propellant, such as by the addition of, for example, one or more cosolvent, antisolvent, solubilizing agent, adjuvant or other propellant modifying material.

Compositions including a combination of a LABA active agent, a LAMA active agent, and an ICS active agent as described herein do not exhibit co-formulation effects relative to compositions including fewer active agents. The lack of a coformulation effect can be assessed by in vivo or in vitro performance characteristics, and is evidenced when the compositions including a LABA, LAMA, and ICS exhibit one or more of FPF, FPM, DDU, AUC₀₋₁₂, and/or Cₘₐₓ characteristics that do not deviate from those exhibited by a similar composition formulated to provide the same delivered dose of the active being evaluated.

In certain embodiments of the compositions described herein, the ICS:LABA delivered dose ratio (i.e., the ratio of ICS delivered dose to LABA delivered dose per actuation of an MDI) is about 5:1 or greater. For example, the ICS:LABA delivered dose ratio may be selected from about 10:1 or greater, about 15:1 or greater, about 20:1 or greater, about 35:1 or greater, and about 50:1 or greater. In further embodiments, the ICS:LAMA delivered dose ratio (i.e., the ratio of ICS delivered dose to LAMA delivered dose per actuation of an MDI) is about 5:1 or greater. For example, the ICS:LAMA delivered dose ratio may be selected from about 10:1 or greater, about 15:1 or greater, about 20:1 or greater, about 35:1 or greater, and about 50:1 or greater. In still further embodiments, compositions as described herein are formulated to provide an ICS:LABA delivered dose ratio selected from about 5:1 or greater, about 10:1 or greater, about 15:1 or greater, about 20:1 or greater, about 35:1 or greater, and about 50:1 or greater and an ICS:LAMA delivered dose ratio selected from about 5:1 or greater, about 10:1 or greater, about 15:1 or greater, about 20:1 or greater, about 35:1 or greater, and about 50:1 or greater.

In specific embodiments, the compositions described herein include a first species of active agent particles comprising formoterol, a second species of active agent particles comprising glycopyrronium, a third species of active agent particles comprising mometasone, suspending particles formed using a phospholipid material, and a suspension medium comprising an HFA propellant, with each of the species of active agent particles and the suspending particles being substantially insoluble in the suspension medium. Compositions according to such embodiments may be formulated to exhibit no coformulation effect as described herein even where the ICS:LAMA delivered dose ratio and/or the ICS:LABA delivered dose ratio is/are selected from about 5:1 or greater, about 10:1 or greater, about 15:1 or greater, about 20:1 or greater, about 35:1 or greater, and about 50:1 or greater. In such embodiments, the composition may comprise sufficient glycopyrronium to provide a delivered dose of less than 10 μg per actuation and sufficient formoterol to provide a delivered dose of less than 5 ug per actuation. In such embodiments, the glycopyrronium may be glycopyrrolate, the formoterol may be formoterol fumarate, and the mometasone may be mometasone furoate. In even more specific embodiments, one, two or all three of the active agents may be provided as a micronized crystalline material, and the suspending particles may be respirable perforated microstructures formed using a phospholipid, such as DSPC. Even further, compositions as described in this paragraph may be formulated to include a ratio of suspending particles to active agent particles selected from between about 0.5:1 and about 75:1, between about 0.5:1 and about 50:1, between about 0.5:1 and about 35:1, between about 0.5:1 and about 25:1, between about 0.5:1 and about 15:1, between about 0.5:1 and about 10:1, between and about 0.5:1 and about 5:1. In alternative such embodiments, the ratio of the total mass of the suspending particles to the total mass of the active agent particles may be selected from between about 1.5:1 and about 75:1, between about 1.5:1 and about 50:1, between about 1.5:1 and about 35:1, between about 1.5:1 and about 25:1, between about 1.5:1 and about 15:1, between about 1.5:1 and about 10:1, and between about 1.5:1 and about 5:1. In further such embodiments, the ratio of the total mass of the suspending particles to the total mass of the active agent particles may be selected from between about 2.5:1 and about 75:1, between about 2.5:1 and about 50:1, between about 2.5:1 and about 35:1, between about 2.5:1 and about 25:1, between about 2.5:1 and about 15:1, between about 2.5:1 and about 10:1, and between about 2.5:1 and about 5:1.

In other specific embodiments, the compositions described herein include a first species of active agent particles comprising formoterol, a second species of active agent particles comprising glycopyrronium, a third species of active agent particles comprising budesonide, suspending particles formed using a phospholipid material, and a suspension medium comprising an HFA propellant, with each of the species of active agent particles and the suspending particles being substantially insoluble in the suspension medium. Compositions according to such embodiments may be formulated to exhibit no coformulation effect as described herein even where the ICS:LAMA delivered dose ratio and/or the ICS:LABA delivered dose ratio is/are selected from about 5:1 or greater, about 10:1 or greater, about 15:1 or greater, about 20:1 or greater, about 35:1 or greater, and about 50:1 or greater. In such embodiments, the composition may comprise sufficient glycopyrronium to provide a delivered dose of less than 10 ug per actuation and sufficient formoterol to provide a delivered dose delivered dose of less than 5 ug per actuation. In such embodiments, the glycopyrronium may be glycopyrrolate and the formoterol may be formoterol fumarate. In even more specific embodiments, one, two or all three of the active agents may be provided as a micronized crystalline material, and the suspending particles may be respirable perforated microstructures formed using a phospholipid, such as DSPC. Even further, compositions as described in this paragraph may be formulated to include a ratio of suspending particles to active agent particles selected from between about 0.5:1 and about 75:1, between about 0.5:1 and about 50:1, between about 0.5:1 and about 35:1, between about 0.5:1 and about 25:1, between about 0.5:1 and about 15:1, between about 0.5:1 and about 10:1, and between about 0.5:1 and about 5:1. In alternative such embodiments, the ratio of the total mass of the suspending particles to the total mass of the active agent particles may be selected from between about 1.5:1 and about 75:1, between about 1.5:1 and about 50:1, between about 1.5:1 and about 35:1, between about 1.5:1 and about 25:1, between about 1.5:1 and about 15:1, between about 1.5:1 and about 10:1, and between about 1.5:1 and about 5:1. In further such embodiments, the ratio of the total mass of the suspending particles to the total mass of the active agent particles may be selected from between about 2.5:1 and about 75:1, between about 2.5:1 and about 50:1, between about 2.5:1 and about 35:1, between about 2.5:1 and about 25:1, between about 2.5:1 and about 15:1, between about 2.5:1 and about 10:1, and between about 2.5:1 and about 5:1.

(iv) Examples of Triple Combination Compositions

Examples of cosuspension compositions suitable for respiratory delivery of a fixed combination of a LABA active agent, a LAMA active agent, and an ICS active agent from an MDI via oral inhalation are provided.

In a first example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 7.5 μg of the pharmaceutically acceptable salt, ester, or isomer of formoterol per actuation of the MDI,

wherein the composition is formulated to provide a delivered dose less than or equal to 10 μg of the pharmaceutically acceptable salt, ester, or isomer of glycopyrronium per actuation of the MDI, and

wherein the ICS:LABA delivered dose ratio is at least 5:1 and the ICS:LAMA delivered dose ratio is at least 5:1.

In a second example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and (v) a plurality of phospholipid suspending particles formed separately from each of the different species of active agent particles,

wherein the composition is formulated to provide a delivered dose less than or equal to 7.5 μg of the pharmaceutically acceptable salt, ester, or isomer of formoterol per actuation of the MDI,

wherein the composition is formulated to provide a delivered dose less than or equal to 10 μg of the pharmaceutically acceptable salt, ester, or isomer of glycopyrronium per actuation of the MDI, and

wherein the ICS:LABA delivered dose ratio is at least 10:1 and the ICS:LAMA delivered dose ratio is at least 7.5:1.

In a third example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 7.5 μg of the pharmaceutically acceptable salt, ester, or isomer of formoterol per actuation of the MDI

wherein the composition is formulated to provide a delivered dose less than or equal to 10 μg of the pharmaceutically acceptable salt, ester, or isomer of glycopyrronium per actuation of the MDI, and

wherein the ICS:LABA delivered dose ratio is at least 15:1 and the ICS:LAMA delivered dose ratio is at least 10:1.

In a fourth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 7.5 μg of the pharmaceutically acceptable salt, ester, or isomer of formoterol per actuation of the MDI,

wherein the composition is formulated to provide a delivered dose less than or equal to 10 μg of the pharmaceutically acceptable salt, ester, or isomer of glycopyrronium per actuation of the MDI, and

wherein the ICS:LABA delivered dose ratio is at least 20:1 and the ICS:LAMA delivered dose ratio is at least 15:1.

In a fifth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 7.5 μg of the pharmaceutically acceptable salt, ester, or isomer of formoterol per actuation of the MDI

wherein the composition is formulated to provide a delivered dose less than or equal to 10 μg of the pharmaceutically acceptable salt, ester, or isomer of glycopyrronium per actuation of the MDI, and

wherein the ICS:LABA delivered dose ratio is at least 25:1 and the ICS:LAMA delivered dose ratio is at least 20:1.

In a sixth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 7.5 μg of the pharmaceutically acceptable salt, ester, or isomer of formoterol per actuation of the MDI,

wherein the composition is formulated to provide a delivered dose less than or equal to 10 μg of the pharmaceutically acceptable salt, ester, or isomer of glycopyrronium per actuation of the MDI, and

wherein the ICS:LABA delivered dose ratio is at least 30:1 and the ICS:LAMA delivered dose ratio is at least 20:1.

In a seventh example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 5.0 μg of the pharmaceutically acceptable salt, ester, or isomer of formoterol per actuation of the MDI,

wherein the composition is formulated to provide a delivered dose less than or equal to 7.5 μg of the pharmaceutically acceptable salt, ester, or isomer of glycopyrronium per actuation of the MDI, and

wherein the ICS:LABA delivered dose ratio is at least 5:1 and the ICS:LAMA delivered dose ratio is at least 5:1.

In an eighth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles, wherein the composition is formulated to provide a delivered dose less than or equal to 5.0 μg of the pharmaceutically acceptable salt, ester, or isomer of formoterol per actuation of the MDI,

wherein the composition is formulated to provide a delivered dose less than or equal to 7.5 μg of the pharmaceutically acceptable salt, ester, or isomer of glycopyrronium per actuation of the MDI, and

wherein the ICS:LABA delivered dose ratio is at least 10:1 and the ICS:LAMA delivered dose ratio is at least 7.5:1.

In a ninth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 5.0 μg of the pharmaceutically acceptable salt, ester, or isomer of formoterol per actuation of the MDI,

wherein the composition is formulated to provide a delivered dose less than or equal to 7.5 μg of the pharmaceutically acceptable salt, ester, or isomer of glycopyrronium per actuation of the MDI, and

wherein the ICS:LABA delivered dose ratio is at least 15:1 and the ICS:LAMA delivered dose ratio is at least 10:1.

In a tenth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 5.0 μg of the pharmaceutically acceptable salt, ester, or isomer of formoterol per actuation of the MDI,

wherein the composition is formulated to provide a delivered dose less than or equal to 7.5 μg of the pharmaceutically acceptable salt, ester, or isomer of glycopyrronium per actuation of the MDI, and

wherein the ICS:LABA delivered dose ratio is at least 20:1 and the ICS:LAMA delivered dose ratio is at least 15:1.

In an eleventh example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of phospholipid suspending particles formed separately from each of the different species of active agent particles,

wherein the composition is formulated to provide a delivered dose less than or equal to 5.0 μg of the pharmaceutically acceptable salt, ester, or isomer of formoterol per actuation of the MDI,

wherein the composition is formulated to provide a delivered dose less than or equal to 7.5 μg of the pharmaceutically acceptable salt, ester, or isomer of glycopyrronium per actuation of the MDI, and

wherein the ICS:LABA delivered dose ratio is at least 25:1 and the ICS:LAMA delivered dose ratio is at least 20:1.

In a twelfth example, the composition includes:

(i) a suspension medium including a pharmaceutically acceptable propellant;

(ii) a first species of respirable active agent particles including a pharmaceutically acceptable salt, ester, or isomer of formoterol;

(iii) a second species of respirable active agent particles including a pharmaceutically acceptable salt, ester, or isomer of glycopyrronium;

(iv) a third species of respirable active agent particles including a pharmaceutically acceptable salt, ester, or isomer of budesonide; and

(v) a plurality of respirable phospholipid suspending particles different formed separately from each of the different species of active agent particles, wherein the composition is formulated to provide a delivered dose less than or equal to 5.0 μg of the pharmaceutically acceptable salt, ester, or isomer of formoterol per actuation of the MDI,

wherein the composition is formulated to provide a delivered dose less than or equal to 7.5 μg of the pharmaceutically acceptable salt, ester, or isomer of glycopyrronium per actuation of the MDI, and

wherein the ICS:LABA delivered dose ratio is at least 30:1 and the ICS:LAMA delivered dose ratio is at least 20:1.

In each of the compositions described herein, including in compositions according to the twelve example compositions, the pharmaceutically acceptable salt, ester, or isomer of formoterol may be formoterol fumarate, and the pharmaceutically acceptable salt, ester, or isomer of glycopyrronium may be the bromide salt of glycopyrronium, namely (3-[(cyclopentylhydroxyphenylacetyl)oxy]-1,1-dimethyl-, bromide).

The compositions described herein, including compositions according to the twelve example compositions, may be provided with an amount of suspending particles that provides a desired ratio of the total mass of the suspending particles to the total mass of the active agent particles. For instance, where the example compositions described herein are formulated to provide an ICS:LABA delivered dose ratio of at least 20:1 and an ICS:LAMA delivered dose ratio of at least 15:1, the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, and between about 0.75:1 and about 2:1. Alternatively, where the example compositions are formulated to provide an ICS:LABA delivered dose ratio of at least 15:1 and a ICS:LAMA delivered dose ratio of at least 10:1, the ratio of the total mass of the suspending particles to the total mass of the active agent particles may be selected from between about 1:1 and about 10:1, such as between about 1:1 and about 7.5:1, between about 1:1 and about 5:1, between about 1:1 and about 2.5:1, between about 2.5:1 and about 10:1, between about 2.5:1 and about 7.5:1, and between about 2.5:1 and 5:1. Further, where the example compositions are formulated to provide an ICS:LABA delivered dose ratio of at least 5:1 and an ICS:LAMA delivered dose ratio of at least 5:1, the ratio of the total mass of the suspending particles to the total mass of the active agent particles may be selected from between about 2:1 and about 15:1, such as between about 1:1 and about 7.5:1, between about 1:1 and about 5:1, between about 1:1 and about 2.5:1, between about 2.5:1 and about 10:1, between about 2.5:1 and about 7.5:1, and between about 2.5:1 and 5:1.

In a specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide)

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 5:1 and the ICS:LAMA delivered dose ratio is at least 5:1, and

wherein the ratio of the total mass of the suspending particles to the total mass of the active agent particles may be selected from between about 2:1 and about 15:1, such as between about 1:1 and about 7.5:1, between about 1:1 and about 5:1, between about 1:1 and about 2.5:1, between about 2.5:1 and about 10:1, between about 2.5:1 and about 7.5:1, or between about 2.5:1 and 5:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 10:1 and the ICS:LAMA delivered dose ratio is at least 7.5:1, and

wherein the ratio of the total mass of the suspending particles to the total mass of the active agent particles may be selected from between about 2:1 and about 15:1, such as between about 1:1 and about 7.5:1, between about 1:1 and about 5:1, between about 1:1 and about 2.5:1, between about 2.5:1 and about 10:1, between about 2.5:1 and about 7.5:1, or between about 2.5:1 and 5:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 15:1 and the ICS:LAMA delivered dose ratio is at least 10:1, and

wherein the ratio of the total mass of the suspending particles to the total mass of the active agent particles may be selected from between about 1:1 and about 10:1, such as between about 1:1 and about 7.5:1, between about 1:1 and about 5:1, between about 1:1 and about 2.5:1, between about 2.5:1 and about 10:1, between about 2.5:1 and about 7.5:1, or between about 2.5:1 and 5:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 20:1 and the ICS:LAMA delivered dose ratio is at least 15:1, and

wherein the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, or between about 0.75:1 and about 2:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 25:1 and the ICS:LAMA delivered dose ratio is at least 20:1, and

wherein the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, or between about 0.75:1 and about 2:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 30:1 and the ICS:LAMA delivered dose ratio is at least 20:1, and

wherein the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, or between about 0.75:1 and about 2:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 7.5 μg per actuation of the MDI and a delivered dose of the pharmaceutically acceptable salt, ester, or isomer of glycopyrronium that is less than or equal to 10 μg per actuation of the MDI,

wherein the ICS:LABA delivered dose ratio is at least 5:1 and the ICS:LAMA delivered dose ratio is at least 5:1, and

wherein the ratio of the total mass of the suspending particles to the total mass of the active agent particles may be selected from between about 2:1 and about 15:1, such as between about 1:1 and about 7.5:1, between about 1:1 and about 5:1, between about 1:1 and about 2.5:1, between about 2.5:1 and about 10:1, between about 2.5:1 and about 7.5:1, or between about 2.5:1 and 5:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 5.0 μg per actuation of the MDI and a delivered dose of the pharmaceutically acceptable salt, ester, or isomer of glycopyrronium that is less than or equal to 7.5 μg per actuation of the MDI,

wherein the ICS:LABA delivered dose ratio is at least 10:1 and the ICS:LAMA delivered dose ratio is at least 7.5:1, and

wherein the ratio of the total mass of the suspending particles to the total mass of the active agent particles may be selected from between about 2:1 and about 15:1, such as between about 1:1 and about 7.5:1, between about 1:1 and about 5:1, between about 1:1 and about 2.5:1, between about 2.5:1 and about 10:1, between about 2.5:1 and about 7.5:1, or between about 2.5:1 and 5:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 5.0 μg per actuation of the MDI and a delivered dose of the pharmaceutically acceptable salt, ester, or isomer of glycopyrronium that is less than or equal to 7.5 μg per actuation of the MDI,

wherein the ICS:LABA delivered dose ratio is at least 15:1 and the ICS:LAMA delivered dose ratio is at least 10:1, and

wherein the ratio of the total mass of the suspending particles to the total mass of the active agent particles may be selected from between about 1:1 and about 10:1, such as between about 1:1 and about 7.5:1, between about 1:1 and about 5:1, between about 1:1 and about 2.5:1, between about 2.5:1 and about 10:1, between about 2.5:1 and about 7.5:1, or between about 2.5:1 and 5:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide I; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 5.0 μg per actuation of the MDI and a delivered dose of the pharmaceutically acceptable salt, ester, or isomer of glycopyrronium that is less than or equal to 7.5 μg per actuation of the MDI,

wherein the ICS:LABA delivered dose ratio is at least 20:1 and the ICS:LAMA delivered dose ratio is at least 15:1, and

wherein the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, or between about 0.75:1 and about 2:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 5.0 μg per actuation of the MDI and a delivered dose of the pharmaceutically acceptable salt, ester, or isomer of glycopyrronium that is less than or equal to 7.5 μg per actuation of the MDI,

wherein the ICS:LABA delivered dose ratio is at least 25:1 and the ICS:LAMA delivered dose ratio is at least 20:1, and

wherein the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, or between about 0.75:1 and about 2:1.

In another specific example, a composition according to the present description includes:

(i) a suspension medium including a pharmaceutically acceptable HFA propellant;

(ii) a first species of respirable active agent particles formed using formoterol fumarate;

(iii) a second species of respirable active agent particles formed using (3-[(cyclopentylhydroxyphenylacetyl)oxy]-1,1-dimethyl-, bromide);

(iv) a third species of respirable active agent particles formed using a pharmaceutically acceptable salt, ester, or isomer of budesonide; and

(v) a plurality of phospholipid suspending particles formed separately from each of the different species of active agent particles,

wherein the composition is formulated to provide a delivered dose of the pharmaceutically acceptable salt, ester, or isomer of formoterol that is less than or equal to 5.0 μg per actuation of the MDI and a delivered dose of the pharmaceutically acceptable salt, ester, or isomer of glycopyrronium that is less than or equal to 7.5 μg per actuation of the MDI,

wherein the ICS:LABA delivered dose ratio is at least 30:1 and the ICS:LAMA delivered dose ratio is at least 20:1, and

wherein the ratio of the total mass of the suspending particles to the total mass of the active agent particles may be selected from between about 0.5:1 and about 5:1, such as between about 0.5:1 and about 3:1, between about 0.5:1 and about 2:1, between about 0.75:1 and about 5:1, between about 0.75:1 and about 3:1, or between about 0.75:1 and about 2:1.

In each of the twelve example compositions and in each of the compositions provided by the specific examples recited herein, the compositions can be formulated to provide a desired delivered dose of the formoterol, glycopyrronium, and budesonide active agents. For example, compositions according to the twelve example compositions and the other specific examples recited herein can be formulated to provide a delivered dose of formoterol, glycopyrronium, and budesonide selected from the combinations of delivered doses defined herein for compositions including budesonide as the ICS active agent, including, e.g., combinations of delivered doses selected from those defined in preceding paragraph [00078].

In each of the compositions described herein, including in each of the twelve example compositions and in each of the compositions provided by the specific examples recited herein, the suspending particles may be provided by or formed using any of the phospholipid materials and associated methods described herein, and the formoterol, glycopyrronium, and budesonide active agents utilized in the three species of active agent particles may be selected from any of the formoterol, glycopyrronium, and budesonide materials described herein (including any combinations thereof). Further, in each of the compositions described herein, each of the active agent particle species and the suspending particles may be selected and/or formulated to be substantially insoluble in the suspension medium. If desired, the materials forming one, more or all of the three different species of active agent particles and the suspending particles may be selected from material(s) that is(are) substantially insoluble, slightly soluble, very slightly soluble, or practically insoluble as defined herein.

In each of the compositions described herein, including in each of the twelve example compositions and in each of the compositions provided by the specific examples recited herein, the respirable suspending particles may be formed using a dry, particulate phospholipid material, such as DSPC. Moreover, the suspending particles, including those formed using DSPC, may be provided as perforated microstructures as described herein. Where DSPC is used as a material for forming the respirable suspending particles, the respirable suspending particles may be formed of a combination of DSPC and CaCl2.

In each of the compositions described herein, including in each of the twelve example compositions and in each of the compositions provided by the specific examples recited herein, one, two or all of the pharmaceutically acceptable salt, ester, or isomer of formoterol, the pharmaceutically acceptable salt, ester, or isomer of glycopyrronium, and the pharmaceutically acceptable salt, ester, or isomer of budesonide may be provided as micronized, crystalline material. For example, in each of the twelve example compositions and in each of the compositions provided by the specific examples recited herein, the first species of active agent particles may be the pharmaceutically acceptable salt, ester, or isomer of formoterol provided as a respirable, micronized crystalline material, the second species of active agent particles may be the pharmaceutically acceptable salt, ester, or isomer of glycopyrronium provided as a respirable, micronized crystalline material, or the third species of active agent particles may be the pharmaceutically acceptable salt, ester, or isomer of budesonide provided as a respirable, micronized crystalline material. Further, in each of the twelve example compositions and in each of the compositions provided by the specific examples recited herein, all of the three species of active agent particles may be provided as a respirable, micronized crystalline material (i.e., the first species of active agent particles may be the pharmaceutically acceptable salt, ester, or isomer of formoterol provided as a respirable, micronized crystalline material, the second species of active agent particles may be the pharmaceutically acceptable salt, ester, or isomer of glycopyrronium provided as a respirable, micronized crystalline material, and the third species of active agent particles may be the pharmaceutically acceptable salt, ester, or isomer of budesonide provided as a respirable, micronized crystalline material).

In each of the compositions described herein, including in each of the twelve example compositions and in each of the compositions provided by the specific examples recited herein, the pharmaceutically acceptable propellant may be an HFA propellant selected from any of the HFA propellants described herein. Moreover, the propellant included in the suspension medium of any of the twelve specified examples or of any of the other specific examples of compositions described herein may be substantially free of a co-solvent or solubilizing agent.

### III. METERED DOSE INHALER SYSTEMS

As described in relation to the methods provided herein, the co-suspension compositions disclosed herein may be used in an MDI system. MDIs are configured to deliver a specific amount of a medicament in aerosol form. In one embodiment, an MDI system includes a pressurized, liquid phase formulation-filled canister disposed in an actuator formed with a mouthpiece. An MDI system according to the present description may include a composition as described herein, which includes a suspension medium, active agent particles providing each of the three or more active agents, and at least one species of suspending particles. The canister used in the MDI may be of any suitable configuration, and in one exemplary embodiment, the canister may have a volume ranging from about 5 mL to about 25 mL, such as, for example a canister having a 19 mL volume. After shaking the device, the mouthpiece is inserted into a patient's mouth between the lips and teeth. The patient typically exhales deeply to empty the lungs and then takes a slow deep breath while actuating the MDI.

Generally, an MDI includes a metering valve having a metering chamber capable of holding a defined volume of the composition to be aerosolized (e.g., 63 μl or any other suitable volume available in commercially available metering valves). The composition is released from the metering chamber into an expansion chamber at the distal end of the valve stem when the MDI is actuated. The actuator of the MDI may be formed to retain the canister containing the composition and may also include a port with an actuator nozzle for receiving the valve stem of the metering valve. When actuated, the specified volume of composition to be aerosolized travels to the expansion chamber, out the actuator nozzle, and into a high-velocity spray that is drawn into the lungs of a patient.

### IV. METHODS

Methods of formulating a pharmaceutical composition for respiratory delivery of a fixed combination of a LABA active agent, a LAMA active agent, and an ICS active agent are provided herein. In one embodiment, the method involves the steps of providing a suspension medium as described herein, providing three or more species of active agent particles, with each species of active agent particle providing a separate active agent, and one or more species of suspending particles, and combining such constituents to form a suspension composition wherein the different species of active agent particles associate with the suspending particles and co-locate with the suspending particles within the suspension medium such that a co-suspension as described herein is formed. In one such embodiment, the association of the different species of active agent particles with the suspending particles is such that they do not separate due to their different buoyancies in a propellant. In certain embodiments, the active agent particles consist essentially of the active agent material, and are free of additional excipients, adjuvants, stabilizers, etc. In specific embodiments, the methods for preparing a co-suspension composition as described herein provide compositions that are suitable for delivery of a fixed combination of a LABA, LAMA, and ICS from an MDI and that do not exhibit a coformulation effect.

Methods for preparing an MDI for respiratory delivery of three or more active agents from the compositions described herein are also disclosed. In certain embodiments, such a method may include loading a canister suitable for use in an inhaler, such as an MDI, with a composition according to the present description. An actuator valve can be attached to an end of the canister and the canister sealed. The actuator valve may be adapted for dispensing a metered amount of the composition (and, as a result, a metered amount of each of the active agents) per actuation of the MDI.

Methods for treating patients suffering from an inflammatory or obstructive pulmonary disease or condition are provided herein. In specific embodiments, such methods include pulmonary delivery of a pharmaceutical composition as described herein, and in certain such embodiments, pulmonary administration of the pharmaceutical composition is accomplished by delivering the composition using an MDI. The disease or condition to be treated can be selected from any inflammatory or obstructive pulmonary disease or condition that responds to the administration of, for example, at least one of a LABA active agent, LAMA active agent, or ICS active agent included in the composition delivered. In particular embodiments, the pharmaceutical compositions described herein may be used in treating a disease or disorder selected from asthma, COPD, exacerbation of airways hyper reactivity consequent to other drug therapy, allergic rhinitis, sinusitis, pulmonary vasoconstriction, inflammation, allergies, impeded respiration, respiratory distress syndrome, pulmonary hypertension, pulmonary vasoconstriction, emphysema, and any other respiratory disease, condition, trait, genotype or phenotype that can respond to the administration of combinations of active agents described herein. In certain embodiments, the pharmaceutical compositions described herein may be used in treating pulmonary inflammation and obstruction associated with cystic fibrosis.

The specific examples included herein are for illustrative purposes only and are not to be considered as limiting to this disclosure. Moreover, the compositions, systems and methods disclosed herein have been described in relation to certain embodiments thereof, and many details have been set forth for purposes of illustration, it will be apparent to those skilled in the art that the invention is susceptible to additional embodiments and that certain of the details described herein may be varied without departing from the basic principles of the invention. Any active agents and reagents used in the following examples are either commercially available or can be prepared according to standard literature procedures by those skilled in the art of organic synthesis. The entire contents of all publications, patents, and patent applications referenced herein are hereby incorporated herein by reference.

### Example 1

Patients diagnosed with COPD may be prescribed inhaled medicines of three distinct classes simultaneously: a beta-agonist; a muscarinic antagonist, and an inhaled corticosteroid. In this example, three different triple cosuspension compositions for respiratory delivery of a combination of a LABA, formoterol fumarate (FF), a LAMA, glycopyrrolate (GPBr), and an ICS, mometasone furoate (MF), were prepared and evaluated. These compositions provided dose-proportional drug delivery for each of three different actives that was observed to be independent of presence of the other active components.

The FF, GPBr, and MF materials were provided as a separate species of active agent particles, with each of the active agents provided as micronized, crystalline material. The co-suspension compositions were provided in pressurized metered dose inhalers (“MDI” or “MDIs”), with each formulation prepared to provide a delivered dose of 4.8 μg/actuation and 18 μg/actuation, respectively, of FF and GPBr. However, the amount of MF included in the three different compositions was varied, with compositions prepared to provide a delivered dose of MF selected from one of 100 μg/actuation, 200 μg/actuation, and 300 μg/actuation.

The suspending particles used in these co-suspensions were perforated microstructures prepared as described herein using a phospholipid material (DSPC). Drug crystals of GPBr (Boehringer Ingelheim, Petersberg, Va.) were micronized by air jet milling after receipt (median size, X₅₀, ˜1.6 μm), but the FF (Inke, S.A., Barcelona Spain; X₅₀˜1.4 μm) and MF (Hovione, Loures Portugal; X₅₀˜1.6 μm) drug crystals were used as received. The Co-suspension formulations were prepared in HFA 134a propellant (Mexichem, S.A., Tlanepantla Mexico) and filled into 14 mL fluorinated ethylene polymer coated aluminum canisters (Presspart, Blackburn, UK), packaged with 50 μl valves, and delivered using an actuator with orifice size 0.3 mm (Bespak, King's Lynn, UK). Aerodynamic particle size distributions (aPSD) were obtained using the Next Generation Impactor (NGI) with the flow rate set to 30±1 LPM (n=3). Drug content was measured using ion-exchange HPLC with UV detection for FF and GP or reversed-phase HPLC with UV detection for MF.

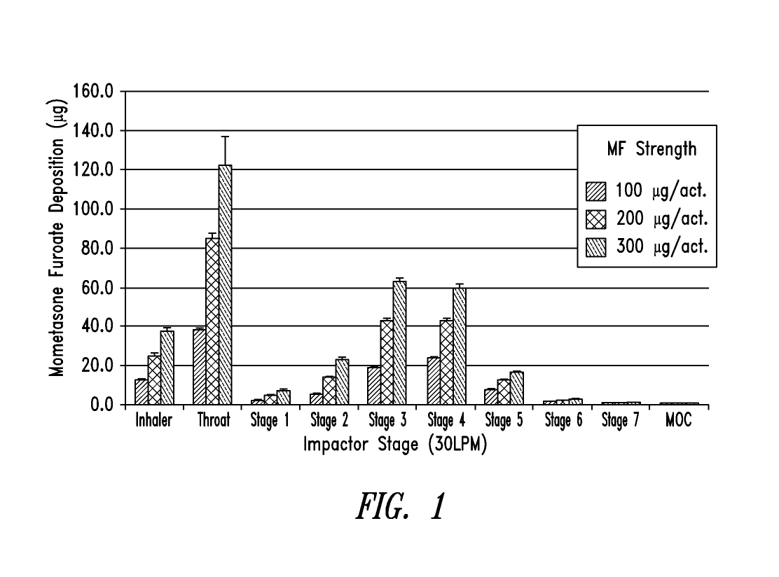

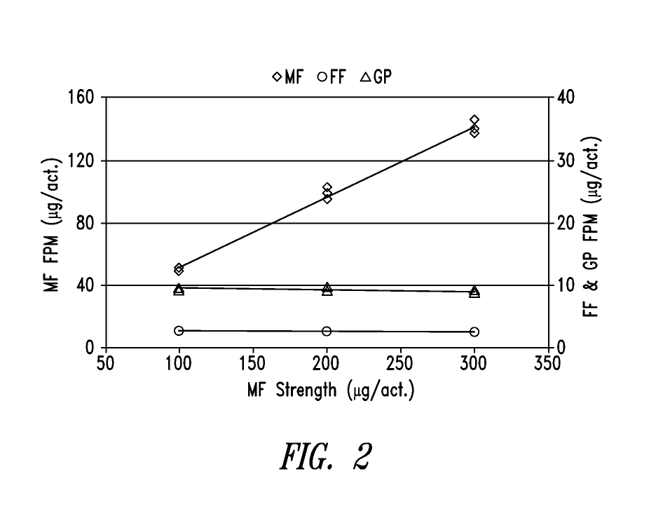

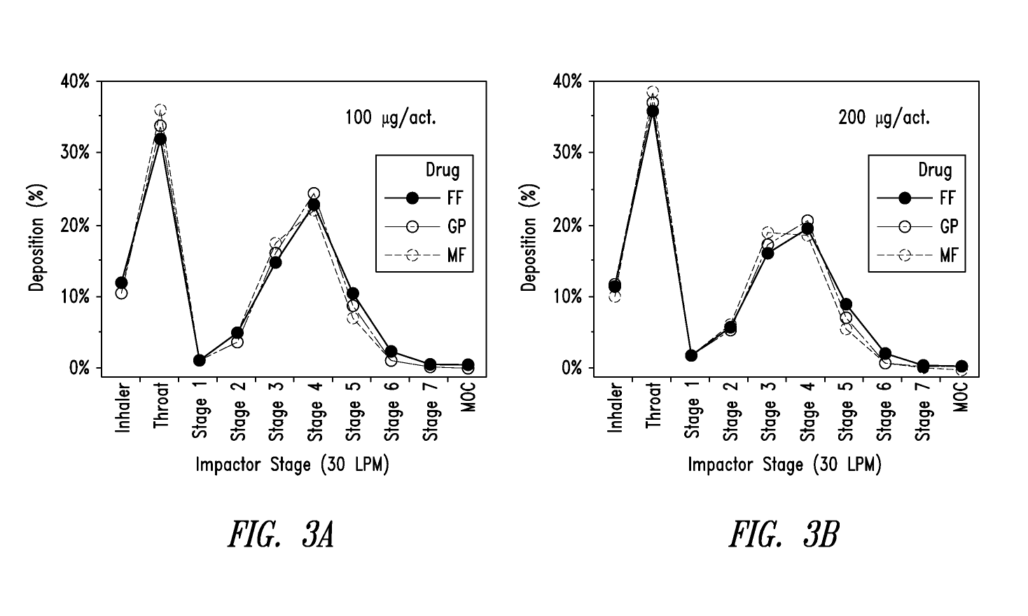

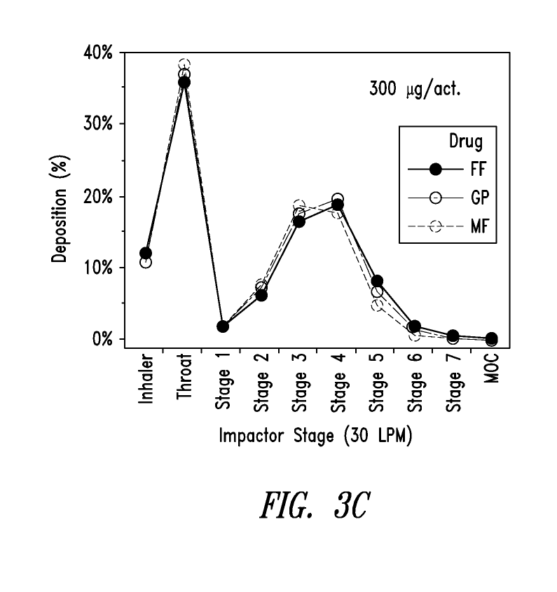

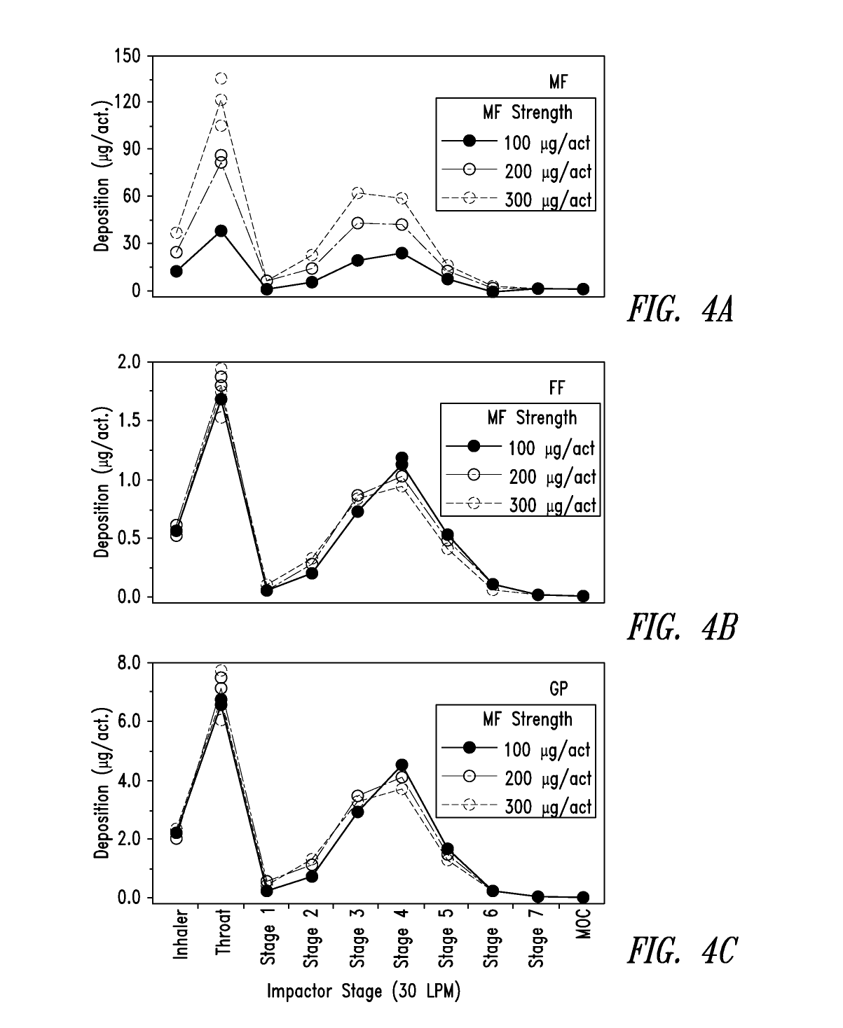

The cascade impaction profiles of the MF across the three triple compositions are shown in FIG. 1. The cascade impaction profiles for each of FF, glycopyrronium (GP), and MF across the different triple compositions are additionally shown in FIG. 3 and FIG. 4. Dose proportionality was observed in all regions of the impactor, demonstrating aerodynamic particle size independent performance across multiple strengths. The fine particle mass (FPM, equal to the sum of drug mass deposited from stages 3 through MOC) for the three drugs is in the three different triple compositions is shown in FIG. 2. The MF FPM showed nearly ideal dose proportionality (r²=0.99, and a slope of 0.48). FPM values for FF and GP remained virtually unchanged when MF strength was varied. Without being bound by a particular theory, it is believed that the similarity in aerosol stage deposition across the various products with increasing strength of the MF was owed, at least in part, to the presence of the phospholipid suspending particles and the formation of particle ensembles as the active agent particles associated with the suspending particles.

Currently, available commercial products of MF have formulations containing 110 and 220 μg per inhalation via inhalation powder, or 100 and 200 μg/actuation via MDI. The cosuspension formulations prepared in this example demonstrate dose proportionality over a 50% broader range of formulation strengths. The aerosol and deliverability characteristics of co-suspension compositions prepared as described herein were similar to those expected from a solution composition for delivery from an MDI, where all the components are generally delivered with the same deposition pattern in the cascade impactor. However, unlike solution-based MDI formulations, the co-suspension compositions described herein facilitate formulation of high dose actives without the need to alter the basic composition, such as by using or increasing the amount of a cosolvent to increase solubility.

### Example 2

Exemplary triple cosuspension compositions deliverable from an MDI were prepared according to the present description. The compositions included a combination of budesonide (BD), glycopyrrolate (GPBr) and formoterol fumarate (FF), with each being provided as a micronized, crystalline material. The micronized BD, GPBr, and FF materials were co-suspended in HFA propellant with suspending particles (SP). The SP used in each MDI cosuspension formulation were spray-dried porous particles formed of 1,2 distearoyl-sn-glycero-3-phosphocholine (DSPC) and calcium chloride (CaCl₂).

Three different triple cosuspension compositions were prepared, with each of the compositions prepared to provide a delivered dose of 9 μg GPBr per MDI actuation and 4.8 μg FF per MDI actuation. The delivered dose of 9 μg GPBr provided a delivered dose of glycopyrronium (GP) of 7.2 μg per MDI actuation. Two of the triple cosuspensions, labeled BFG1 and BGF2, were formulated to provide a delivered dose of 160 μg BD per MDI actuation. A third triple cosuspension composition was formulated to provide a delivered dose of 40 μg BD per MDI actuation. Information regarding the materials cosuspended within the HFA propellant in each of the three triple cosuspension compositions is provided in Table 1.

In addition to the exemplary triple cosuspension compositions, a mono cosuspension composition including only BD as an active agent (BD Mono), a dual cosuspension including a combination of GPBr and FF (GFF), and a dual cosuspension including a combination of BD and FF (BFF) were prepared. Information regarding the materials cosuspended in the HFA propellant for the BD Mono, GFF, and BFF compositions is provided in Table 1. The BD Mono composition was formulated to provide a delivered dose of 160 μg BD per MDI actuation. The GFF composition was formulated to provide a delivered dose of 7.2 μg GP per MDI actuation and a delivered dose of 4.8 μg FF per MDI actuation. The BFF composition was formulated to provide a delivered dose of 160 μg BD per MDI actuation and a delivered dose of 4.8 μg FF per MDI actuation.

TABLE 1

| | | Delivered Dose per MDI |
|---|---|---|
| | SP | Actuation |
| Formulation | Concentration | (BD/GP/FF) |
| BGF 1 (N-1325-010A) | 3.0 mg/ml | 160 μg/ 7.2 μg/4.8 μg |
| BGF 2 (N-1333-010A) | 3.0 mg/ml | 160 μg/7.2 μg/4.8 μg |
| BGF 3 (F079) | 5.85 mg/ml | 40 μg/7.2 μg/4.8 μg |
| GFF (F078) | 5.85 mg/ml | —/7.2 μg/4.8 μg |
| BFF (N-1429-009) | 4.5 mg/ml | 160 μg/—/4.8 μg |
| BD Mono (4HO27A) | 5.85 mg/ml | 160 μg/—/— |

For MDI manufacturing, a drug addition vessel (DAV) was prepared for suspension filling in the following manner. All powders were weighed into a drug addition vessel (DAV) within a nitrogen purged glove box that is controlled to <5% RH, by first adding half of the SP quantity, next filling the microcrystalline active agent material(s), and lastly adding the remaining half of the SP to the top. The DAV was sealed, removed from the glove box, and connected to the suspension vessel. The powders were rinsed into the vessel with HFA. The suspension was stirred and recirculated for no less than 60 minutes before MDI filling commenced. Product was formulated to a target fill weight of 10.8±0.5 g/canister. The temperature inside the suspension vessel was maintained at 15-17° C. throughout batch production. After recirculation for 30 min the cosuspension compositions were filled into 14 mL fluorinated ethylene polymer (FEP) coated aluminum canisters (Presspart, Blackburn, UK) through commercially available metering valves (Bespak, King's Lynn, UK). Sample canisters were then selected from each batch fill for total canister analysis to ensure that formulation targets were met.

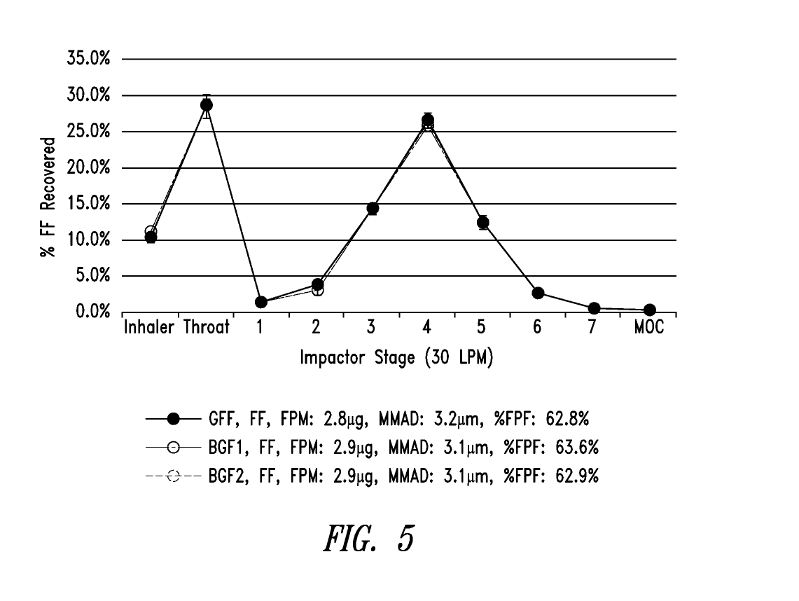

The aerosol performance of each of the triple cosuspension compositions was evaluated and compared to the aerosol performance provided by the GFF, BFF, and BD Mono cosuspensions. Aerosol performance and aerodynamic particle size distributions were obtained using the Next Generation Impactor (NGI) with the flow rate set to 30±1 LPM (n=3). FIG. 5-FIG. 8 illustrate results of the evaluation and comparisons.

FIG. 5 provides cascade impaction profiles for FF provided by the BGF1, BGF2, and GFF cosuspension compositions. The particle size distribution of the micronized, crystalline BD material included in BGF1 was relatively coarse (X₉₀ of 3.34 μm, based on primary particle size as measured by Sympatec) when compared to that of the BD material included in BFG2 (X₉₀ of 2.99 μm, based on primary particle size as measured by Sympatec). The concentration of SP included in BGF1 and BGF2 was 3.0 mg/ml, while that of the GFF cosuspension was 5.85 mg/ml, and the GFF cosuspension contained no BD, while the BGF1 and BGF2 compositions were both formulated to provide a delivered dose of 160 μg BD per MDI actuation. Despite differences in BD particle size distribution, and significant differences in both the concentration of BD and the concentration of SP included in the different cosuspension compositions, no coformulation effect was observed for FF when formulated in the exemplary triple cosuspensions. As can be appreciated by reference to FIG. 5, the cascade impaction profiles for FF and the FPM, MMAD and FPF for FF were nearly identical for each of the BGF1, BGF2, and GFF compositions.

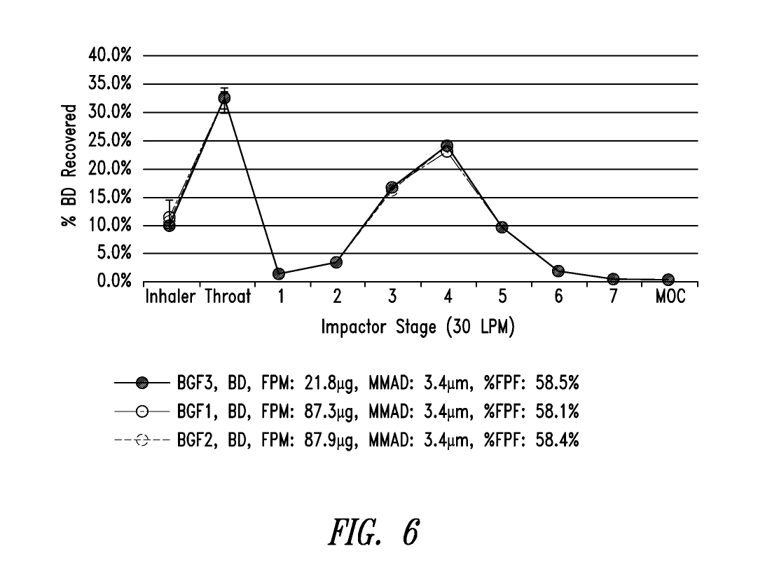

FIG. 6 provides cascade impaction profiles for BD provided by the BGF1, BGF2, and BFG3 cosuspensions. The particle size distribution of the micronized, crystalline BD material included in BGF1 was again relatively coarse compared to that of the crystalline BD material included in BFG2. The concentration of SP included in BGF1 and BGF2 was 3.0 mg/ml, while that of the BGF3 cosuspension was 5.85 mg/ml. Moreover, the BGF1 and BFG2 compositions provided a delivered dose of 160 μg BD per MDI actuation, while BGF3 provided a delivered dose of 40 μg BD per MDI actuation. Despite such differences among the compositions, no coformulation effect was observed for BD when formulated in the exemplary triple cosuspensions. As can be appreciated by reference to FIG. 6, the cascade impaction profiles for BD and the FPM, MMAD and FPF for BD were nearly identical for each of the BGF1, BGF2, and BGF3 compositions.

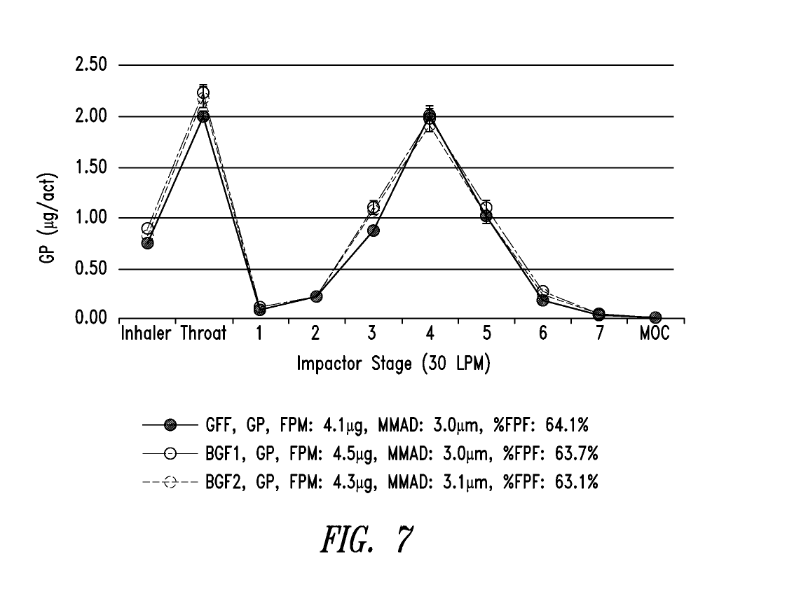

FIG. 7 provides cascade impaction profiles for GP provided by the BGF1, BGF2, and GFF cosuspensions. Again, the particle size distribution of the micronized, crystalline BD material included in BGF1 was relatively coarse compared to that of the crystalline BD material included in BFG2. The concentration of SP included in BGF1 and BGF2 was 3.0 mg/ml, while that of the GFF cosuspension was 5.85 mg/ml. Moreover, BGF1 and BFG2 provided a delivered dose of 160 μg BD per MDI actuation, while the GFF composition included no BD. Despite such differences among the compositions, no coformulation effect was observed for GP when formulated in the exemplary triple cosuspensions. As can be appreciated by reference to FIG. 7, the cascade impaction profiles for GP and the FPM, MMAD and FPF for GP were nearly identical for each of the BGF1, BGF2, and GFF compositions.

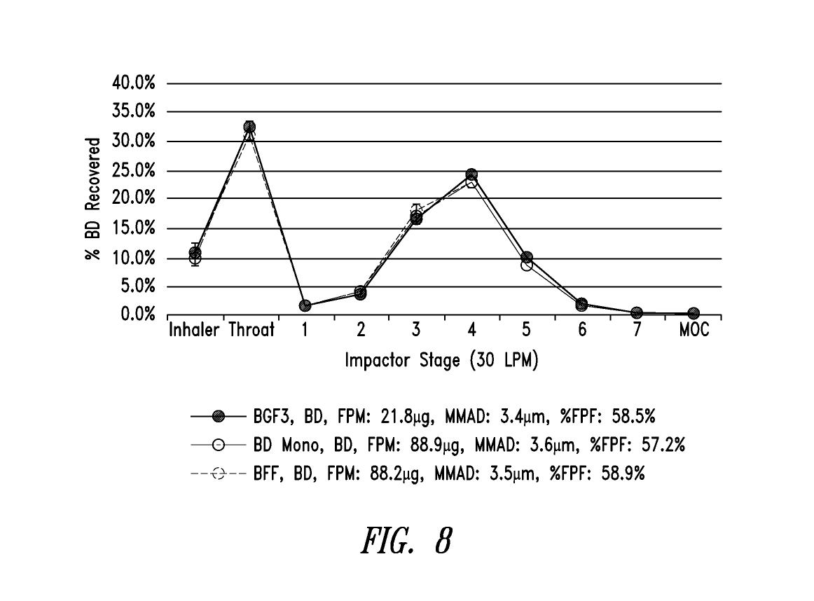

FIG. 8 provides cascade impaction profiles for BD provided by the BGF3, BD Mono, and BFF cosuspensions. The concentration of SP included in BGF3 and BD Mono was 5.85 mg/ml, while that of the BFF cosuspension was 4.5 mg/ml. The BD Mono and BFF compositions provided a delivered dose of 160 μg BD per MDI actuation, while BGF3 provided a delivered dose of 40 μg BD per MDI actuation. Moreover, BGF3 included GPBr and FF, while BFF did not include GPBr and BD Mono did not include GPBr or FF. Despite the differences between the compositions, no coformulation effect was observed for BD when formulated in the exemplary triple cosuspensions. As can be appreciated by reference to FIG. 8, the cascade impaction profiles for BD and the FPM, MMAD and FPF for BD were nearly identical for each of the BD Mono, BFF, and BGF3 compositions.

### Example 3

A double-blind, four-period, six-treatment, single-dose, cross-over clinical study in healthy adult volunteers was conducted to evaluate three different triple cosuspension compositions prepared according to the present description. In particular, the pharmacokinetic (PK) performance and safety of the exemplary triple cosuspensions was evaluated.

Three different triple cosuspension compositions including BD, GPBr, and FF were prepared. Each of the active agents was provided as a micronized crystalline material, and the micronized BD, GPBr, and FF materials were co-suspended in a hydrofluoroalkane (HFA) propellant with suspending particles (SP). The HFA propellant used was HFA 134a, and the SP used in each MDI cosuspension formulation were spray-dried porous particles formed of 1,2 distearoyl-sn-glycero-3-phosphocholine (DSPC) and calcium chloride (CaCl₂). The triple cosuspension compositions (approximately 10.8 g in the finished product) were filled into 14 mL fluorinated ethylene polymer (FEP) coated aluminum canisters (Presspart, Blackburn, UK) through commercially available metering valves (Bespak, King's Lynn, UK).

Each of the three triple cosuspension compositions included SP at a concentration of 5.85 g/ml. Further, each of the three compositions were formulated to provide a delivered dose of 7.2 μg GP per MDI actuation and a delivered dose of 4.8 μg FF per MDI actuation. However, the amount of BD included in each of the triple cosuspension compositions was adjusted to provide compositions providing different strengths of BD. In the first triple cosuspension (BGF 160), the composition was formulated to provide a delivered dose of 160 μg BD per MDI actuation. In the second triple cosuspension (BGF 80), the composition was formulated to provide a delivered dose of 80 μg BD per MDI actuation, and in the third triple cosuspension (BGF 40), the composition was formulated to provide a delivered dose of 40 μg BD per MDI actuation.

The BGF MDI compositions were administered as two inhalations twice daily (BID) by oral inhalation. The corresponding doses of GP and FF for each strength of the triple cosuspension were 14.4 μg and 9.6 μg per administration, respectively, yielding total doses of 28.8 μg GP and 19.2 μg FF per day. The corresponding doses of BD for each of the prepared triple cosuspensions were 320 μg (BGF 160), 160 μg (BGF 80), and 80 μg (BGF 40) per administration, yielding total doses of 640 μg (BGF 160), 320 μg (BGF 80), and 160 μg (BGF 40) per day.

After priming, each canister delivers 7.2 μg/4.8 μg GP/FF per actuation from the actuator (delivered dose) and 8.3 μg/5.5 μg GP/FF per actuation from the valve (metered dose). The corresponding deliveries for BD are 160 μg (BGF 160), 80 μg (BGF 80), and 40 μg (BGF 40) per actuation from the actuator and 185.0 μg (BGF 160), 92.5 μg (BGF 80), and 46.2 μg (BGF 40) per actuation from the valve. It should be noted that 4.8 μg FF ex-actuator is equivalent to 5.0 μg ex-actuator formoterol fumarate dihydrate. In addition to the three active ingredients, each actuation of the MDIs containing the triple cosuspension compositions delivered approximately 262 μg of SP and 63 mg of HFA-134a from the actuator.

The GFF dual cosuspension composition was prepared similarly to the triple cosuspension compositions, except that the GFF composition included no BD. Micronized, crystalline GPBr and FF were cosuspended in HFA 134a with spray-dried porous particles formed of 1,2 distearoyl-sn-glycero-3-phosphocholine (DSPC) and calcium chloride (CaCl2) as the SP. The GFF composition included the SP at a concentration of 5.85 mg/ml, and the GFF composition was formulated to provide a delivered dose of 7.2 μg GP per MDI actuation and a delivered dose of 4.8 μg FF per MDI actuation. The GFF composition was administered as two inhalations twice daily (BID) by oral inhalation. The corresponding doses of GP and FF were 14.4 μg and 9.6 μg per administration, respectively, yielding total doses of 28.8 μg GP and 19.2 μg FF per day.

Following determination of study eligibility, 84 subjects were randomized to one of 12 treatment sequences balanced for period and first order carry over effect. The study was conducted at a single clinical research center in the United States. PK measurements and safety assessments were performed prior to dosing and for 12 hours post-dose.

Bioequivalence was determined by comparing the 90% CI for the geometric mean ratio (GMR) to bounds of 80% to 125% for BD and FF. Due to the high variability of GP, for purposes of assessing bioequivalence, bounds of 67% to 150% were used in combination with the requirement that the point estimate for the GMR lie between 80% and 125%.

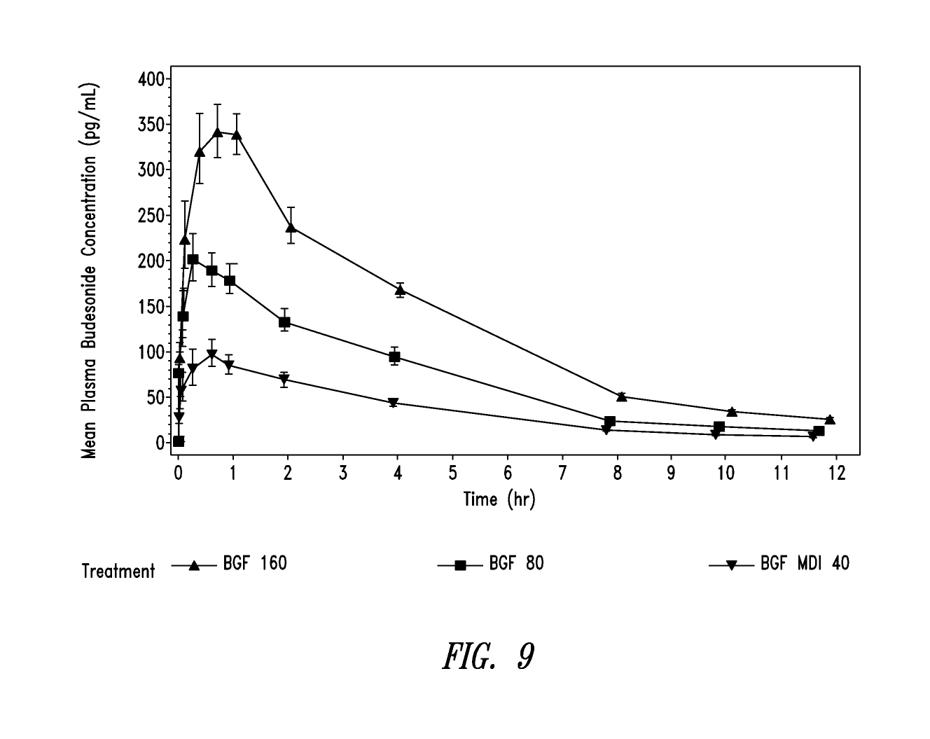

FIG. 9 provides the geometric mean plasma concentration-time profile of BD by treatment following single dose administration in healthy volunteers in the clinical study. As is shown in FIG. 9, there was a near linear relationship between BGF MDI dose and systemic exposure.

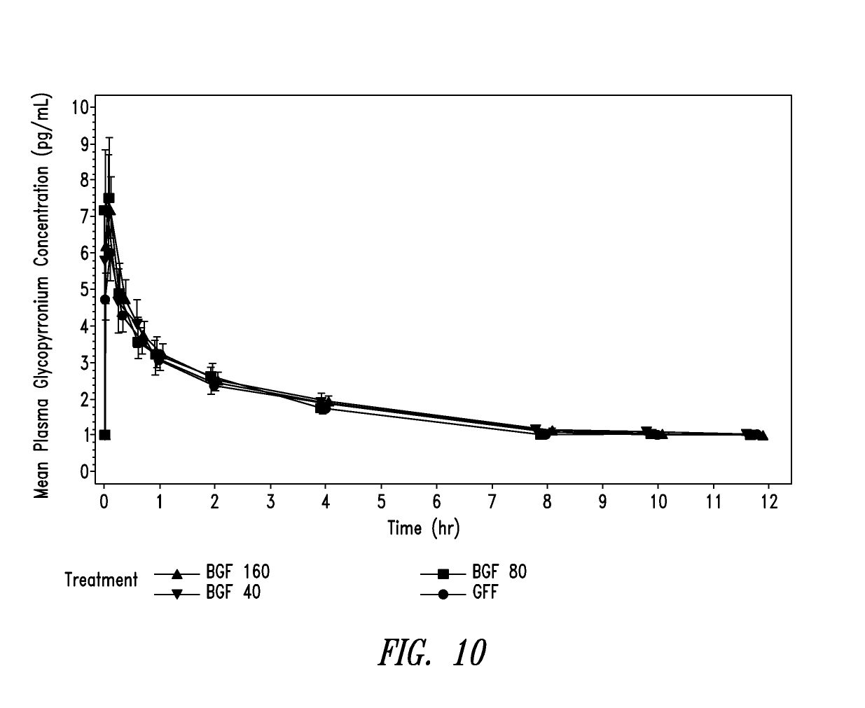

FIG. 10 provides the geometric mean plasma concentration-time profile of GP by treatment following single dose administration in the clinical study. The GP dose was the same across all treatments, and the plasma concentration profiles show consistent results. Based on the comparisons of the triple cosuspension treatments with GFF, it was concluded that the presence of BD in the compositions did not meaningfully impact the GP systemic exposure. For BGF 160, both the AUC₀₋₁₂ and Cₘₐₓ comparisons met the pre-specified bioequivalence bounds of 67% to 150% with point estimates within 80% to 125%. It is worth noting that, for AUC₀₋₁₂, the 90% CI for the GMR fell within traditional bioequivalence bounds of 80% to 125%.

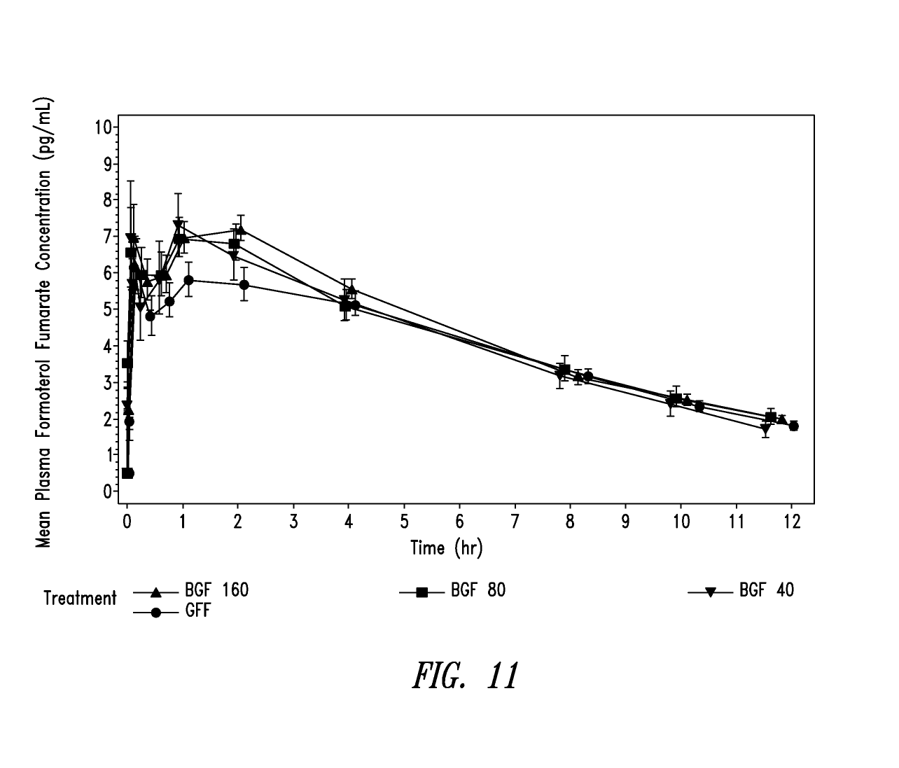

FIG. 11 provides the geometric mean plasma concentration-time profile of FF by treatment following single dose administration in healthy volunteers in the clinical study. The FF systemic exposure was similar across all triple cosuspension treatments compared to each other and compared to GFF.

Based on the comparisons of the triple cosuspension treatments to GFF, it was concluded that the presence of BD in the compositions did not meaningfully impact the exposure levels of FF. For all three strengths of the triple cosuspensions, both the AUC₀₋₁₂ and Cₘₐₓ comparisons achieved bioequivalence compared to GFF. For instance, the GMR for BGF 160 compared to GFF was 1.04 (0.97, 1.11) for AUC₀₋₁₂ and 1.11 (1.01, 1.22) for Cₘₐₓ. Bioequivalence was also achieved for GP and FF following administration of BGF 160 compared to the systemic exposure following administration of GFF. These results support that the addition of BD to GFF in the triple cosuspension compositions did not meaningfully impact the exposure levels of BD and did not give rise to a coformulation effect. All treatment arms were well tolerated with a low frequency of adverse events, and no untoward safety signals were observed.

1. A suspension composition for respiratory delivery of a long-acting muscarinic antagonist (LAMA), a long-acting β₂ adrenergic agonist (LABA), and an inhaled corticosteroid (ICS) from a metered dose inhaler (MDI) to a patient, the composition comprising:
a suspension medium comprising a pharmaceutically acceptable propellant;
a first species of respirable active agent particles comprising the LABA active agent that is substantially insoluble in the suspension medium;
a second species of respirable active agent particles comprising the LAMA active agent that is substantially insoluble in the suspension medium;
a third species of respirable active agent particles comprising the ICS active agent that is substantially insoluble in the suspension medium;
a plurality of respirable suspending particles, wherein the plurality of suspending particles are formed of a material that is substantially insoluble in the suspension medium, and the ICS active agent and LABA active agent are included in the suspension composition such that the ICS:LABA delivered dose ratio is at least 5:1 per actuation of the MDI.

2. The suspension composition of claim 1, wherein the LABA active agent is selected from bambuterol, clenbuterol, formoterol, salmeterol, carmoterol, milveterol, indacaterol, and saligenin- or indole-containing and adamantyl-derived β₂ agonists.

3. The suspension composition of claim 2, wherein the LABA active agent is a pharmaceutically acceptable salt, ester, or isomer of formoterol selected from hydrochloric, hydrobromic, sulfuric, phosphoric, fumaric, maleic, acetic, lactic, citric, tartaric, ascorbic, succinic, glutaric, gluconic, tricarballylic, oleic, benzoic, p-methoxybenzoic, salicylic, o- and p-hydroxybenzoic, p-chlorobenzoic, methanesulfonic, p-toluenesulfonic and 3-hydroxy-2-naphthalene carboxylic acid salts.

4. The suspension composition of claim 3, wherein the pharmaceutically acceptable salt of formoterol is formoterol fumarate.

5. The suspension composition of claim 1, wherein the LAMA active agent is selected from glycopyrronium, dexipirronium, scopolamine, tropicamide, pirenzepine, dimenhydrinate, tiotropium, darotropium, aclidinium, trospium, ipatropium, atropine, benzatropin, and oxitropium.

6. The suspension composition of claim 5, wherein the LAMA active agent is a pharmaceutically acceptable salt, ester, or isomer of glycopyrronium selected from fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate, and benzenesulfonate salts

7. The suspension composition of claim 6, wherein the pharmaceutically acceptable glycopyrronium salt is selected from fluoride, chloride, bromide, and iodide salts.

8. The suspension composition of claim 7, wherein the pharmaceutically acceptable salt of glycopyrronium is 3-[(cyclopentyl-hydroxyphenylacetyl)oxy]-1,1-dimethylpyrrolidinium bromide which has the following structure:

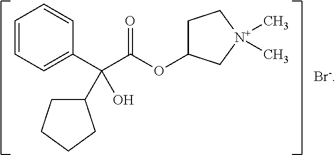

9. The suspension composition of claim 1, wherein the ICS active agent is selected from beclomethasone, budesonide, ciclesonide, flunisolide, fluticasone, methyl-prednisolone, mometasone, prednisone, and triamcinolone.

10. The suspension composition of claim 9, wherein the ICS active agent is selected from a pharmaceutically acceptable salt, ester, or isomer of mometasone or budesonide.

11. The suspension composition of claim 10, wherein the ICS active agent is budesonide.

12. The suspension composition according to claim 1, wherein at least one of the first, second, and third species of active agent particle comprises a micronized crystalline material.

13. The suspension composition according to claim 1, wherein the first species of active agent particle comprises respirable, crystalline particles of the LABA active agent.

14. The suspension composition according to claim 1, wherein the second species of active agent particle comprises respirable, crystalline particles of the LAMA active agent.

15. The suspension composition according to claim 1, wherein the third species of active agent particle comprises respirable, crystalline particles of the ICS active agent.

16. The suspension composition according to claim 12, wherein the first species of active agent particle comprises respirable, crystalline particles of the LABA active agent, the second species of active agent particle comprises respirable, crystalline particles of the LAMA active agent, and the third species of active agent particle comprises respirable, crystalline particles of the ICS active agent.

17. The suspension composition according to claim 1, wherein the ICS active agent and LABA active agent are included in the suspension composition such that the ICS:LABA delivered dose ratio per actuation of the MDI is selected from about 10:1 or greater, about 15:1 or greater, about 20:1 or greater, about 35:1 or greater, and about 50:1 or greater.

18. The suspension composition according to claim 17, wherein the first species of active agent particles comprises a pharmaceutically acceptable salt, ester, or isomer of formoterol selected from hydrochloric, hydrobromic, sulfuric, phosphoric, fumaric, maleic, acetic, lactic, citric, tartaric, ascorbic, succinic, glutaric, gluconic, tricarballylic, oleic, benzoic, p-methoxybenzoic, salicylic, o- and p-hydroxybenzoic, p-chlorobenzoic, methanesulfonic, p-toluenesulfonic and 3-hydroxy-2-naphthalene carboxylic acid salts, and the third species of active agent particles comprises a pharmaceutically acceptable salt, ester, or isomer of mometasone or budesonide.

19. The suspension composition according to claim 18, wherein the first species of active agent particles comprises formoterol fumarate, the third species of active agent particles comprises a pharmaceutically acceptable salt, ester, or isomer of budesonide, and the ICS active agent and LABA active agent are included in the suspension composition such that the ICS:LABA delivered dose ratio per actuation of the MDI is selected from about 5:1 or greater, 10:1 or greater, about 15:1 or greater, about 20:1 or greater, about 35:1 or greater, and about 50:1.

20. The suspension composition according to claim 18, wherein the first species of active agent particles comprises formoterol fumarate, the third species of active agent particles comprises a pharmaceutically acceptable salt, ester, or isomer of mometasone furoate, and the ICS active agent and LABA active agent are included in the suspension composition such that the ICS:LABA delivered dose ratio per actuation of the MDI is selected from about 10:1 or greater, about 15:1 or greater, about 20:1 or greater, about 35:1 or greater, and about 50:1.

21. The suspension composition according to claim 1, wherein the ICS active agent and LAMA active agent are included in the suspension composition such that the ICS:LAMA delivered dose ratio per actuation of the MDI is selected from about 5:1 or greater, about 10:1 or greater, about 15:1 or greater, about 20:1 or greater, about 35:1 or greater, and about 50:1 or greater.

22. The suspension composition according to claim 21, wherein the second species of active agent particles comprise a pharmaceutically acceptable salt, ester, or isomer of glycopyrronium selected from fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate, and benzenesulfonate salts, and the third species of active agent particles comprise a pharmaceutically acceptable salt, ester, or isomer of mometasone or budesonide.

23. The suspension composition according to claim 22, wherein the wherein the pharmaceutically acceptable glycopyrronium salt is selected from fluoride, chloride, bromide, and iodide salts, and the third species of active agent particles comprise a pharmaceutically acceptable salt, ester, or isomer of budesonide.

24. The suspension composition of claim 23, wherein the pharmaceutically acceptable salt of glycopyrronium is 3-[(cyclopentyl-hydroxyphenylacetyl)oxy]-1,1-dimethylpyrrolidinium bromide, and the third species of active agent particles comprise a pharmaceutically acceptable salt, ester, or isomer of budesonide.

25. The suspension composition according to claim 23, wherein the ICS active agent and LABA active agent are included in the suspension composition such that the ICS:LABA delivered dose ratio per actuation of the MDI is selected from about 5:1 or greater, 10:1 or greater, about 15:1 or greater, about 20:1 or greater, about 35:1 or greater, and about 50:1.

26. The suspension composition according to claim 22, wherein the third species of active agent particles comprises a pharmaceutically acceptable salt, ester, or isomer of mometasone, and the ICS active agent and LABA active agent are included in the suspension composition such that the ICS:LABA delivered dose ratio per actuation of the MDI is selected from about 10:1 or greater, about 15:1 or greater, about 20:1 or greater, about 35:1 or greater, and about 50:1.

27. The suspension composition according to claim 1, wherein a ratio of total mass of the suspending particles to total mass of the first, second, and third active agent particles is selected from between about 0.5:1 and about 75:1, between about 0.5:1 and about 50:1, between about 0.5:1 and about 35:1, between about 0.5:1 and about 25:1, between about 0.5:1 and about 15:1, between about 0.5:1 and about 10:1, and between about 0.5:1 and about 5:1.

28. The suspension composition according to claim 1, wherein a ratio of total mass of the suspending particles to total mass of the first, second, and third active agent particles is selected from between about 1.5:1 and about 75:1, between about 1.5:1 and about 50:1, between about 1.5:1 and about 35:1, about 1.5:1 and about 25:1, about 1.5:1 and about 15:1, about 1.5:1 and about 10:1, and between about 1.5:1 and about 5:1.

29. The suspension composition according to claim 1, wherein a ratio of total mass of the suspending particles to total mass of the first, second, and third active agent particles is selected from between about 2.5:1 and about 75:1, between about 2.5:1 and about 50:1, between about 2.5:1 and about 35:1, between about 2.5:1 and about 25:1, between about 2.5:1 and about 15:1, between about 2.5:1 and about 10:1, and between about 2.5:1 and about 5:1.

30. The suspension composition of claim 27, wherein the first species of active agent particles comprises a pharmaceutically acceptable salt, ester, or isomer of formoterol, the second species of active agent particles comprises a pharmaceutically acceptable salt, ester, or isomer of glycopyrronium, the third species of active agent particles comprises a pharmaceutically acceptable salt, ester, or isomer of budesonide, the ICS active agent and LABA active agent are included in the suspension composition such that the ICS:LABA delivered dose ratio per actuation of the MDI is selected from about 5:1 or greater, about 10:1 or greater, about 15:1 or greater, about 20:1 or greater, about 35:1 or greater, and about 50:1, and the ICS active agent and LABA active agent are included in the suspension composition such that the ICS:LABA delivered dose ratio per actuation of the MDI is selected from about 5:1 or greater, about 10:1 or greater, about 15:1 or greater, about 20:1 or greater, about 35:1 or greater, and about 50:1.

31. The suspension composition of claim 28, wherein the first species of active agent particles comprises a pharmaceutically acceptable salt, ester, or isomer of formoterol, the second species of active agent particles comprises a pharmaceutically acceptable salt, ester, or isomer of glycopyrronium, the third species of active agent particles comprises a pharmaceutically acceptable salt, ester, or isomer of budesonide, the ICS active agent and LABA active agent are included in the suspension composition such that the ICS:LABA delivered dose ratio per actuation of the MDI is selected from about 5:1 or greater, about 10:1 or greater, about 15:1 or greater, about 20:1 or greater, about 35:1 or greater, and about 50:1, and the ICS active agent and LABA active agent are included in the suspension composition such that the ICS:LABA delivered dose ratio per actuation of the MDI is selected from about 5:1 or greater, about 10:1 or greater, about 15:1 or greater, about 20:1 or greater, about 35:1 or greater, and about 50:1.

32. The suspension composition of claim 29, wherein the first species of active agent particles comprises a pharmaceutically acceptable salt, ester, or isomer of formoterol, the second species of active agent particles comprises a pharmaceutically acceptable salt, ester, or isomer of glycopyrronium, the third species of active agent particles comprises a pharmaceutically acceptable salt, ester, or isomer of budesonide, the ICS active agent and LABA active agent are included in the suspension composition such that the ICS:LABA delivered dose ratio per actuation of the MDI is selected from about 5:1 or greater, about 10:1 or greater, about 15:1 or greater, about 20:1 or greater, about 35:1 or greater, and about 50:1, and the ICS active agent and LABA active agent are included in the suspension composition such that the ICS:LABA delivered dose ratio per actuation of the MDI is selected from about 5:1 or greater, about 10:1 or greater, about 15:1 or greater, about 20:1 or greater, about 35:1 or greater, and about 50:1.

33. The suspension composition according to claim 1, wherein the suspending particles comprise dry particulate, perforated microstructures.

34. The suspension composition according to claim 1, wherein the suspending particles comprise 1,2-distearoyl-sn-Glycero-3-phosphocholine (DSPC).

35. The suspension composition according to claim 34, wherein the suspending particles comprise DSPC and calcium chloride.

36. The suspension composition according to claim 1, wherein the pharmaceutically acceptable propellant comprises an HFA propellant.

37. The suspension composition according to claim 36, wherein the suspension medium comprises a pharmaceutically acceptable HFA propellant substantially free of co-solvents and solubilizing agents.

38-67. (canceled)
